SEQUENCE LISTING (137 sequences)

SEQ ID NO: 1 - protein, 106 aa, synthetic construct
  REGION 1..106: note = kappa chain constant domain (light chain constant domain of chimeric recptor)
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 97 aa, synthetic construct
  REGION 1..97: note = gamma immunoglobulin heavy chain (heavy chain constant domain of chimeric receptor)
STKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 3 - protein, 20 aa, synthetic construct
  REGION 1..20: note = spacer, human IgG1 hinge
AEPKSPDKTHTCPPCPKDPK

SEQ ID NO: 4 - protein, 15 aa, synthetic construct
  REGION 1..15: note = spacer, hinge spacer
EPKSCDKTHTCPPCP

SEQ ID NO: 5 - protein, 6 aa, synthetic construct
  REGION 1..6: note = heavy chain variable region (VH) complementarity determining region (CDR), CDR1
SDYAWN

SEQ ID NO: 6 - protein, 16 aa, synthetic construct
  REGION 1..16: note = VH CDR sequence, CDR2
NIWYSGSTTYNPSLKS

SEQ ID NO: 7 - skipped

SEQ ID NO: 8 - protein, 17 aa, synthetic construct
  REGION 1..17: note = light chain variable region (VL) CDR sequence, CDR1
RASESVDSYGKTFMHWH

SEQ ID NO: 9 - protein, 7 aa, synthetic construct
  REGION 1..7: note = VL CDR sequence, CDR2
RVSNLES

SEQ ID NO: 10 - protein, 9 aa, synthetic construct
  REGION 1..9: note = VL CDR sequence, CDR3
QQSNEDPFT

SEQ ID NO: 11 - protein, 112 aa, synthetic construct
  REGION 1..112: note = VH sequence from murine monoclonal antibody
EVQLQESGPGLVKPSQSLSLTCTVTGYSITSDYAWNWIRQFPGNKLEWMGNIWYSGSTTYNPSLKSRISITRDTSKNQFFLQLNSVTSEDTATYYCSRMDFWGQGTTLTVSS

SEQ ID NO: 12 - protein, 112 aa, synthetic construct
  REGION 1..112: note = VL sequence from murine monoclonal antibody
DIVLTQSPPSLAVSLGQRATISCRASESVDSYGKTFMHWHQQKPGQPPKLLIYRVSNLESGIPARFSGSGSRTDFTLTINPVEADDVATYYCQQSNEDPFTFGGGTKLEIKR

SEQ ID NO: 13 - protein, 245 aa, synthetic construct
  REGION 1..245: note = mouse anti-cynomolgus (Macaca fascicularis) CD79b 10D10 scFv sequence
DIVLTQSPPSLAVSLGQRATISCRASESVDSYGKTFMHWHQQKPGQPPKLLIYRVSNLESGIPARFSGSGSRTDFTLTINPVEADDVATYYCQQSNEDPFTFGGGTKLEIKRSGGGGSGGGGSGGGGSGGGGSEVQLQESGPGLVKPSQSLSLTCTVTGYSITSDYAWNWIRQFPGNKLEWMGNIWYSGSTTYNPSLKSRISITRDTSKNQFFLQLNSVTSEDTATYYCSRMDFWGQGTTLTVSS

SEQ ID NO: 14 - protein, 5 aa, synthetic construct
  REGION 1..5: note = VH CDR sequence, CDR1
SYWIE

SEQ ID NO: 15 - protein, 17 aa, synthetic construct
  REGION 1..17: note = VH CDR sequence, CDR2
EILPGGGDTNYNEIFKG

SEQ ID NO: 16 - protein, 8 aa, synthetic construct
  REGION 1..8: note = VH CDR sequence, CDR3
RVPVYFDY

SEQ ID NO: 17 - protein, 15 aa, synthetic construct
  REGION 1..15: note = VL CDR sequence, CDR1
KASQSVDYDGDSFLN

SEQ ID NO: 18 - protein, 7 aa, synthetic construct
  REGION 1..7: note = VL CDR sequence, CDR2
AASNLES

SEQ ID NO: 19 - protein, 9 aa, synthetic construct
  REGION 1..9: note = VL CDR sequence, CDR3
QQSNEDPLT

SEQ ID NO: 20 - protein, 117 aa, synthetic construct
  REGION 1..117: note = VH sequence from murine monoclonal antibody
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPVYFDYWGQGTLVTVSS

SEQ ID NO: 21 - protein, 112 aa, synthetic construct
  REGION 1..112: note = VL sequence from murine monoclonal antibody
DIQLTQSPSSLSASVGDRVTITCKASQSVDYDGDSFLNWYQQKPGKAPKLLIYAASNLESGVPSRFSGSGSGTDFTLTISSLQPEDFATYYCQQSNEDPLTFGQGTKVEIKR

SEQ ID NO: 22 - protein, 270 aa, synthetic construct
  REGION 1..270: note = humanised anti-CD79b-v17 scFv
METDTLLLWVLLLWVPGSTGDIQLTQSPSSLSASVGDRVTITCKASQSVDYDGDSFLNWYQQKPGKAPKLLIYAASNLESGVPSRFSGSGSGTDFTLTISSLQPEDFATYYCQQSNEDPLTFGQGTKVEIKRSGGGGSGGGGSGGGGSGGGGSEVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPVYFDYWGQGTLVTVSS

SEQ ID NO: 23 - protein, 8 aa, synthetic construct
  REGION 1..8: note = VH CDR sequence, CDR3
RVPIRLDY

SEQ ID NO: 24 - protein, 117 aa, synthetic construct [sequence omitted]

SEQ ID NO: 25 - protein, 250 aa, synthetic construct
  REGION 1..250: note = humanised anti-CD79b v18 scFv sequence
DIQLTQSPSSLSASVGDRVTITCKASQSVDYDGDSFLNWYQQKPGKAPKLLIYAASNLESGVPSRFSGSGSGTDFTLTISSLQPEDFATYYCQQSNEDPLTFGQGTKVEIKRSGGGGSGGGGSGGGGSGGGGSEVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSS

SEQ ID NO: 26 - protein, 15 aa, synthetic construct
  REGION 1..15: note = VL CDR sequence, CDR1
KASQSVDYEGDSFLN

SEQ ID NO: 27 - protein, 112 aa, synthetic construct
  REGION 1..112: note = VL sequence from murine monoclonal antibody
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKR

SEQ ID NO: 28 - protein, 270 aa, synthetic construct
  REGION 1..270: note = humanised anti-CD79b v28 scFv sequence
METDTLLLWVLLLWVPGSTGDIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRSGGGGSGGGGSGGGGSGGGGSEVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSS

SEQ ID NO: 29 - protein, 15 aa, synthetic construct
  REGION 1..15: note = VL CDR sequence, CDR1
KASQSVDYSGDSFLN

SEQ ID NO: 30 - protein, 112 aa, synthetic construct
  REGION 1..112: note = VL sequence from murine monoclonal antibody
DIQLTQSPSSLSASVGDRVTITCKASQSVDYSGDSFLNWYQQKPGKAPKLFIYAASNLESGVPSRFSGSGSGTDFTLTISSLQPEDFATYYCQQSNEDPLTFGQGTKVEIKR

SEQ ID NO: 31 - protein, 270 aa, synthetic construct
  REGION 1..270: note = humanised anti-CD79b v32 scFv sequence
METDTLLLWVLLLWVPGSTGDIQLTQSPSSLSASVGDRVTITCKASQSVDYSGDSFLNWYQQKPGKAPKLFIYAASNLESGVPSRFSGSGSGTDFTLTISSLQPEDFATYYCQQSNEDPLTFGQGTKVEIKRSGGGGSGGGGSGGGGSGGGGSEVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSS

SEQ ID NO: 32 - protein, 117 aa, synthetic construct
  REGION 1..117: note = VH sequence from murine monoclonal antibody
EVQLQQSGAELMKPGASVKISCKATGYTFSSYWIEWVKQRPGHGLEWIGEILPGGGDTNYNEIFKGKATFTADTSSNTAYMQLSSLTSEDSAVYYCTRRVPVYFDYWGQGTSVTVSS

SEQ ID NO: 33 - protein, 112 aa, synthetic construct
  REGION 1..112: note = VL sequence from murine monoclonal antibody
DIVLTQSPASLAVSLGQRATISCKASQSVDYDGDSFLNWYQQKPGQPPKLFIYAASNLESGIPARFSGSGSGTDFTLNIHPVEEEDAATYYCQQSNEDPLTFGAGTELELKR

SEQ ID NO: 34 - protein, 270 aa, synthetic construct
  REGION 1..270: note = murine anti-CD79b SN8 scFv sequence
METDTLLLWVLLLWVPGSTGDIVLTQSPASLAVSLGQRATISCKASQSVDYDGDSFLNWYQQKPGQPPKLFIYAASNLESGIPARFSGSGSGTDFTLNIHPVEEEDAATYYCQQSNEDPLTFGAGTELELKRSGGGGSGGGGSGGGGSGGGGSEVQLQQSGAELMKPGASVKISCKATGYTFSSYWIEWVKQRPGHGLEWIGEILPGGGDTNYNEIFKGKATFTADTSSNTAYMQLSSLTSEDSAVYYCTRRVPVYFDYWGQGTSVTVSS

SEQ ID NO: 35 - protein, 5 aa, synthetic construct
  REGION 1..5: note = VH CDR sequence, CDR3
YAMDY

SEQ ID NO: 36 - protein, 113 aa, synthetic construct
  REGION 1..113: note = VH sequence from murine monoclonal antibody
QVQLQQSGPELVKPGASVKISCKASGYTFTNYGMNWVKQRPGQGLQWIGRIYPGSGSTNYQKFKGKATLTVDKSSSTAYMELRSLTSENSAVYYCARYAMDYTGQGTSVTVSS

SEQ ID NO: 37 - protein, 113 aa, synthetic construct
  REGION 1..113: note = VL sequence from murine monoclonal antibody
DVLMTQTPLSLPVSLGDQASISCRSSQSIVHSNGNTYLEWYLQKPGQSPKLLIYKVSNRPSGVPNRFSGSGSGTDFTLKISRVQAQNLGVYYCFQGSHVPWTFGGGTKLEIKR

SEQ ID NO: 38 - protein, 267 aa, synthetic construct
  REGION 1..267: note = murine anti-CD79a scFv sequence
METDTLLLWVLLLWVPGSTGDVLMTQTPLSLPVSLGDQASISCRSSQSIVHSNGNTYLEWYLQKPGQSPKLLIYKVSNRPSGVPNRFSGSGSGTDFTLKISRVQAQNLGVYYCFQGSHVPWTFGGGTKLEIKRSGGGGSGGGGSGGGGSGGGGSQVQLQQSGPELVKPGASVKISCKASGYTFTNYGMNWVKQRPGQGLQWIGRIYPGSGSTNYQKFKGKATLTVDKSSSTAYMELRSLTSENSAVYYCARYAMDYTGQGTSVTVSS

SEQ ID NO: 39 - protein, 119 aa, synthetic construct
  REGION 1..119: note = VH sequence from 1D9-3 antibody
EVQLVESGGGLVQPKGSLKLSCAASGFTFNTYAMHWVRQAPGKGLEWVARIRSKSSNYATYYADSVKDRFTISRDDSQSMLYLQMNNLKTEDTAMYYCVVDYLYAMDYWGQGTSVTVSS

SEQ ID NO: 40 - protein, 107 aa, synthetic construct
  REGION 1..107: note = VL sequence from 1D9-3 antibody
DIVMTQSQKFMSTSVGDRVSITCKASQNVRTAVAWYQQKPGQSPKALIYLASNRHTGVPDRFTGSGSGTDFTLTISNVQSEDLADYFCLQHWNYPFTFGSGTKLEIK

SEQ ID NO: 41 - protein, 120 aa, synthetic construct
  REGION 1..120: note = VH sequence from 3B4-13 antibody
QVQLQQSGAELVRPGASVTLSCKASGYTFTDYEMHWVKQTPVHGLEWIGAIDPETGATAYNQKFKGKAILTADKSSSTAYMDLRSLTSEDSAVYYCTRYDYGSSPWFAYWGQGTLVTVSA

SEQ ID NO: 42 - protein, 109 aa, synthetic construct
  REGION 1..109: note = VL sequence from 3B4-13 antibody
QAVVTQESALTTSPGETVTLTCRSSAGAVTTSNYANWVQEKPDHLFTGLIGGTNNRAPGVPARFSGSLIGDKAALTITGAQTEDEAIYFCALWNSNHWVFGGGTKLTVL

SEQ ID NO: 43 - protein, 119 aa, synthetic construct
  REGION 1..119: note = VH sequence from 7G6-6 antibody
QVQLQQPGAELVMPGASVKLSCKASGYTFTSYWMHWVKQRPGQGLEWIGEIDPSDSYTNYNQKFKGKATLTVDKSSSTAYMQLSSLTSEDSAVYYCARGYYGSSSFDYWGQGTTLTVSS

SEQ ID NO: 44 - protein, 111 aa, synthetic construct
  REGION 1..111: note = VL sequence from 7G6-6 antibody
DIVMSQSPSSLAVSVGEKVTMSCKSSQSLLYSSNQKNYLAWYQQKPGQSPKLLIYWASTRESGVPDRFTGSGSGTDFTLTISSVKAEDLAVYYCQQYYSYTFGGGTKLEIK

SEQ ID NO: 45 - protein, 120 aa, synthetic construct
  REGION 1..120: note = VH sequence from 6C4-6 antibody
QVQLKESGPGLVAPSQSLSITCTVSGFSLTSYGVHWVRQPPGKGLEWLVVIWSDGSTTYNSALKSRLSISKDNSKSQVFLKMNSLQTDDTAMYYCARHADDYGFAWFAYWGQGTLVTVSA

SEQ ID NO: 46 - protein, 107 aa, synthetic construct
  REGION 1..107: note = VL sequence from 6C4-6 antibody
DIQMTQSPASLSASVGETVTITCRASENIYSYLAWYQQKQGKSPQLLVYNAKTLAEGVPSRFSGSGSGTQFSLKINSLQPEDFGSYYCQHHYGTPPTFGGGTKLEIK

SEQ ID NO: 47 - protein, 121 aa, synthetic construct
  REGION 1..121: note = VH sequence from 4D9-12 antibody
EFQLQQSGPELVKPGASVKISCKASGYSFTDYNMNWVKQSNGKSLEWIGVINPNYGTTSYNQKFKGKATLTVDQSSSTAYMQLNSLTSEDSAVYYCARSSTTVVDWYFDVWGTGTTVTVSS

SEQ ID NO: 48 - protein, 107 aa, synthetic construct
  REGION 1..107: note = VL sequence from 4D9-12 antibody
DIQMTQSPSSLSASLGERVSLTCRASQEISGYLSWLQQKPDGTIKRLIYAASTLDSGVPKRFSGSRSGSDYSLTISSLESEDFADYYCLQYASYPFTFGSGTKLEIK

SEQ ID NO: 49 - protein, 118 aa, synthetic construct
  REGION 1..118: note = VH sequence from 5H4-9 antibody
QVQVQQPGAELVRPGTSVKLSCKASGYTFTRYWMYWVKQRPGQGLEWIGVIDPSDNFTYYNQKFKGKATLTVDTSSSTAYMQLSSLTSEDSAVYYCARGYGSSYVGYWGQGTTLTVSS

SEQ ID NO: 50 - protein, 113 aa, synthetic construct
  REGION 1..113: note = VL sequence from 5H4-9 antibody
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPWTFGGGTKLEIK

SEQ ID NO: 51 - protein, 124 aa, synthetic construct
  REGION 1..124: note = VH sequence from 10C1-D9 antibody
QVTLKESGPGILQSSQTLSLTCSFSGFSLSTSDMGVSWIRQPSGKGLEWLAHIYWDDDKRYNPSLKSRLTISKDASRNQVFLKIATVDTADTATYYCARSPWIYYGHYWCFDVWGTGTTVTVSS

SEQ ID NO: 52 - protein, 107 aa, synthetic construct
  REGION 1..107: note = VL sequence from 10C1-D9 antibody
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPFTFGSGTKLEIK

SEQ ID NO: 53 - protein, 123 aa, synthetic construct
  REGION 1..123: note = VH sequence from 15G7-2 antibody
QVQLQQSGAELVKPGASVKLSCKASGYTFTEYTIHWVKQRSGQGLEWIGWFYPGSGSIKYNEKFKDKATLTADKSSSTVYMELSRLTSEDSAVYFCARHGDGYYLPPYYFDYWGQGTTLTVSS

SEQ ID NO: 54 - protein, 106 aa, synthetic construct
  REGION 1..106: note = VL sequence from 15G7-2 antibody
QIVLTQSPAIMSASPGEKVTMTCSASSSVSYMYWYQQKPGSSPRLLIYDTSNLASGVPVRFSGSGSGTSYSLTISRMEAEDAATYYCQQWSSYPLTFGAGTKLELK

SEQ ID NO: 55 - protein, 121 aa, synthetic construct
  REGION 1..121: note = VH sequence from 2B12-8 antibody
QVQLQQSGAELARPGASVKLSCKASGYIFTSYGISWVKQRTGQGLEWIGEIYPRSGNTYYNEKFKGKATLTADKSSSTAYMELRSLTSEDSAVYFCARPIYYGSREGFDYWGQGTTLTVSS

SEQ ID NO: 56 - protein, 107 aa, synthetic construct
  REGION 1..107: note = VL sequence from 2B12-8 antibody
DIVLTQSPATLSVTPGDSVSLSCRASQSISTNLHWYQQKSHASPRLLIKYASQSVSGIPSRFSGSGSGTDFTLSINSVETEDFGIFFCQQSYSWPYTFGGGTKLEIK

SEQ ID NO: 57 - protein, 120 aa, synthetic construct
  REGION 1..120: note = VH sequence from 2C4-4 antibody
QVQLQQPGAELVMPGASVKLSCKASGYTFTSYWMHWVKQRPGQGLEWIGEIDPSDSYTNYNQKFKGKSTLTVDKSSSTAYIQLSSLTSEDSAVYYCARWASYRGYAMDYWGQGTSVTVSS

SEQ ID NO: 58 - protein, 112 aa, synthetic construct
  REGION 1..112: note = VL sequence from 2C4-4 antibody
DVLMTQTPLSLPVSLGDQASISCRSSQSIVHSNGNTYLEWYLQKPGQSPKLLIYKVSNRFSGVPDRFSGSESGTDFTLKISRVEAEDLGVYYCFQGSHVPWTFGGGTKLEIK

SEQ ID NO: 59 - protein, 119 aa, synthetic construct
  REGION 1..119: note = VH sequence from 3E10-7 antibody
EFQLQQSGPELVKPGASVKISCKASGYSFTDYNMNWVKQSNGKSLEWIGVINPNYGTTSYNQRFKGKATLTVDQSSSTAYMQLNSLTSEDSAVYYCARSGLRYWYFDVWGTGTTVTVSS

SEQ ID NO: 60 - protein, 107 aa, synthetic construct; identical to SEQ ID NO: 48
  REGION 1..107: note = VL sequence from 3E10-7 antibody

SEQ ID NO: 61 - protein, 120 aa, synthetic construct
  REGION 1..120: note = VH sequence from LT22 antibody
EVQLVESGAEVKKPGSSVKVSCKASGYTFTNYWINWVRQAPGQGLEWMGNIYPSDSFTNYNQKFKDRVTITADKSTSTVYLELRNLRSDDTAVYYCTRDTQERSWYFDVWGQGTLVTVSS

SEQ ID NO: 62 - protein, 113 aa, synthetic construct
  REGION 1..113: note = VL sequence from LT22 antibody
DIVMTQSPATLSVSPGERATLSCRSSQSLVHSNGNTYLHWYQQKPGQAPRLLIYKVSNRFSGVPARFSGSGSGAEFTLTISSLQSEDFAVYYCSQSTHVPWTFGQGTRLEIKR

SEQ ID NO: 63 - protein, 121 aa, synthetic construct
  REGION 1..121: note = VH sequence from Inotuzumab G5_44 antibody
EVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSS

SEQ ID NO: 64 - protein, 113 aa, synthetic construct
  REGION 1..113: note = VL sequence from Inotuzumab G5_44 antibody
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKR

SEQ ID NO: 65 - protein, 127 aa, synthetic construct
  REGION 1..127: note = VH sequence from 9A8-1 antibody
EVQLVESGGGLVQPGRSLKLSCAASGFTFSNFAMAWVRQPPTKGLEWVASISTGGGNTYYRDSVKGRFTISRDDAKNTQYLQMDSLRSEDTATYYCARQRNYYDGSYDYEGYTMDAWGQGTSVTVSS

SEQ ID NO: 66 - protein, 107 aa, synthetic construct
  REGION 1..107: note = VL sequence from 9A8-1 antibody
DIQMTQSPSSLSASLGDRVTITCRSSQDIGNYLTWFQQKVGRSPRRMIYGAIKLEDGVPSRFSGSRSGSDYSLTISSLESEDVADYQCLQSIQYPFTFGSGTKLEIK

SEQ ID NO: 67 - protein, 226 aa, Homo sapiens
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 68 - protein, 229 aa, Homo sapiens
MARLALSPVPSHWMVALLLLLSAEPVPAARSEDRYRNPKGSACSRIWQSPRFIARKRGFTVKMHCYMNSASGNVSWLWKQEMDENPQQLKLEKGRMEESQNESLATLTIQGIRFEDNGIYFCQQKCNNTSEVYQGCGTELRVMGFSTLAQLKQRNTLKDGIIMIQTLLIILFIIVPIFLLLDKDDSKAGMEEDHTYEGLDIDQTATYEDIVTLRTGEVKWSVGEHPGQE

SEQ ID NO: 69 - protein, 7 aa, synthetic construct
  REGION 1..7: note = VH CDR sequence, CDR1
GYAFSSS

SEQ ID NO: 70 - protein, 6 aa, synthetic construct
  REGION 1..6: note = VH CDR sequence, CDR2
YPGDED

SEQ ID NO: 71 - protein, 10 aa, synthetic construct
  REGION 1..10: note = VH CDR sequence, CDR3
SLLYGDYLDY

SEQ ID NO: 72 - protein, 10 aa, synthetic construct
  REGION 1..10: note = VL CDR sequence, CDR1
SASSSVSYMH

SEQ ID NO: 73 - protein, 7 aa, synthetic construct
  REGION 1..7: note = VL CDR sequence, CDR2
DTSKLAS

SEQ ID NO: 74 - protein, 9 aa, synthetic construct
  REGION 1..9: note = VL CDR sequence, CDR3
QQWNINPLT

SEQ ID NO: 75 - protein, 119 aa, synthetic construct
  REGION 1..119: note = VH sequence from murine monoclonal antibody
QVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSS

SEQ ID NO: 76 - protein, 107 aa, synthetic construct
  REGION 1..107: note = VL sequence from murine monoclonal antibody
QIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKR

SEQ ID NO: 77 - protein, 241 aa, synthetic construct
  REGION 1..241: note = VH-VL scFv sequence from murine monoclonal antibody
QVQLQQSGPELVKPGASVKISCKASGYAFSSSWMNWVKQRPGKGLEWIGRIYPGDEDTNYSGKFKDKATLTADKSSTTAYMQLSSLTSEDSAVYFCARSLLYGDYLDYWGQGTTLTVSSGGGGSGGGGSGGGGSQIVLTQSPAIMSASPGEKVTMTCSASSSVSYMHWYQQKSGTSPKRWIYDTSKLASGVPDRFSGSGSGTSYFLTINNMEAEDAATYYCQQWNINPLTFGAGTKLELKR

SEQ ID NO: 78 - protein, 5 aa, synthetic construct
  REGION 1..5: note = VH CDR sequence, CDR1
SYWMN

SEQ ID NO: 79 - protein, 16 aa, synthetic construct
  REGION 1..16: note = VH CDR sequence, CDR2
QIWPGDGDTNYNGKFK

SEQ ID NO: 80 - protein, 15 aa, synthetic construct
  REGION 1..15: note = VH CDR sequence, CDR3
RETTTVGRYYYAMDY

SEQ ID NO: 81 - protein, 15 aa, synthetic construct
  REGION 1..15: note = VL CDR sequence, CDR1
KASQSVDYDGDSYLN

SEQ ID NO: 82 - protein, 7 aa, synthetic construct
  REGION 1..7: note = VL CDR sequence, CDR2
DASNLVS

SEQ ID NO: 83 - protein, 9 aa, synthetic construct
  REGION 1..9: note = VL CDR sequence, CDR3
QQSTEDPWT

SEQ ID NO: 84 - protein, 235 aa, synthetic construct
  REGION 1..235: note = Murine CD19ALAb scFv sequence
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSSDIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 85 - protein, 236 aa, synthetic construct
  REGION 1..236: note = Humanised CD19ALAb scFv sequence - Heavy 19, Kappa 16
QVQLVQSGAEVKKPGASVKLSCKASGYAFSSYWMNWVRQAPGQSLEWIGQIWPGDGDTNYNGKFKGRATLTADESARTAYMELSSLRSGDTAVYFCARRETTTVGRYYYAMDYWGKGTLVTVSSDIQLTQSPDSLAVSLGERATINCKASQSVDYDGDSYLNWYQQKPGQPPKLLIYDASNLVSGVPDRFSGSGSGTDFTLTISSLQAADVAVYHCQQSTEDPWTFGQGTKVEIKR

SEQ ID NO: 86 - protein, 236 aa, synthetic construct
  REGION 1..236: note = Humanised CD19ALAb scFv sequence - Heavy 19, Kappa 7
QVQLVQSGAEVKKPGASVKLSCKASGYAFSSYWMNWVRQAPGQSLEWIGQIWPGDGDTNYNGKFKGRATLTADESARTAYMELSSLRSGDTAVYFCARRETTTVGRYYYAMDYWGKGTLVTVSSDIQLTQSPDSLAVSLGERATINCKASQSVDYDGDSYLNWYQQKPGQPPKVLIYDASNLVSGVPDRFSGSGSGTDFTLTISSLQAADVAVYYCQQSTEDPWTFGQGTKVEIKR

SEQ ID NO: 87 - protein, 124 aa, synthetic construct
  REGION 1..124: note = Murine CD19ALAb VH sequence
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTTVTVSS

SEQ ID NO: 88 - protein, 124 aa, synthetic construct
  REGION 1..124: note = Humanised CD19ALAb VH sequence
QVQLVQSGAEVKKPGASVKLSCKASGYAFSSYWMNWVRQAPGQSLEWIGQIWPGDGDTNYNGKFKGRATLTADESARTAYMELSSLRSGDTAVYFCARRETTTVGRYYYAMDYWGKGTLVTVSS

SEQ ID NO: 89 - protein, 111 aa, synthetic construct
  REGION 1..111: note = Murine CD19ALAb VL sequence
DIQLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 90 - protein, 112 aa, synthetic construct
  REGION 1..112: note = Humanised CD19ALAb VL sequence, Kappa 16
DIQLTQSPDSLAVSLGERATINCKASQSVDYDGDSYLNWYQQKPGQPPKLLIYDASNLVSGVPDRFSGSGSGTDFTLTISSLQAADVAVYHCQQSTEDPWTFGQGTKVEIKR

SEQ ID NO: 91 - protein, 112 aa, synthetic construct
  REGION 1..112: note = Humanised CD19ALAb VL sequence, Kappa 7
DIQLTQSPDSLAVSLGERATINCKASQSVDYDGDSYLNWYQQKPGQPPKVLIYDASNLVSGVPDRFSGSGSGTDFTLTISSLQAADVAVYYCQQSTEDPWTFGQGTKVEIKR

SEQ ID NO: 92 - protein, 20 aa, synthetic construct
  REGION 1..20: note = cleavage site, 2A-like sequence
RAEGRGSLLTCGDVEENPGP

SEQ ID NO: 93 - protein, 5 aa, synthetic construct
  REGION 1..5: note = VH CDR sequence, CDR1
NFAMA

SEQ ID NO: 94 - protein, 17 aa, synthetic construct
  REGION 1..17: note = VH CDR sequence, CDR2
SISTGGGNTYYRDSVKG

SEQ ID NO: 95 - protein, 18 aa, synthetic construct
  REGION 1..18: note = VH CDR sequence, CDR3
QRNYYDGSYDYEGYTMDA

SEQ ID NO: 96 - protein, 11 aa, synthetic construct
  REGION 1..11: note = VL CDR sequence, CDR1
RSSQDIGNYLT

SEQ ID NO: 97 - protein, 7 aa, synthetic construct
  REGION 1..7: note = VL CDR sequence, CDR2
GAIKLED

SEQ ID NO: 98 - protein, 7 aa, synthetic construct
  REGION 1..7: note = VL CDR sequence, CDR3
LQSIQYP

SEQ ID NO: 99 - protein, 124 aa, synthetic construct
  REGION 1..124: note = VH sequence from 1G3-4 antibody
QVTLKESGPGILQPSQTLSLTCTFSGFSLSTSGMGVGWIRQPSGKGLEWLTNIWWDDDKNYNPSLKNRLTISKDTSINQAFLKITNVDTADTATYYCARIAHYFDGYYYVMDVWGQGTSVTVSS

SEQ ID NO: 100 - protein, 107 aa, synthetic construct
  REGION 1..107: note = VL sequence from 1G3-4 antibody
DIQMTQSPASLSASLGETVSIECLASGGISNDLAWYQQKSGKSPQLLIYAASRLQDGVPSRFSGSGSGTRYSLKISGMQSEDEADYFCQQSYKYPYTFGGGTKLELK

SEQ ID NO: 101 - protein, 7 aa, synthetic construct
  REGION 1..7: note = VH CDR sequence, CDR1
TSGMGVG

SEQ ID NO: 102 - protein, 16 aa, synthetic construct
  REGION 1..16: note = VH CDR sequence, CDR2
NIWWDDDKNYNPSLKN

SEQ ID NO: 103 - protein, 14 aa, synthetic construct
  REGION 1..14: note = VH CDR sequence, CDR3
IAHYFDGYYYVMDV

SEQ ID NO: 104 - protein, 11 aa, synthetic construct
  REGION 1..11: note = VL CDR sequence, CDR1
LASGGISNDLA

SEQ ID NO: 105 - protein, 7 aa, synthetic construct
  REGION 1..7: note = VL CDR sequence, CDR2
AASRLQD

SEQ ID NO: 106 - protein, 8 aa, synthetic construct
  REGION 1..8: note = VL CDR sequence, CDR3
QQSYKYPY

SEQ ID NO: 107 - protein, 117 aa, synthetic construct
  REGION 1..117: note = VH sequence from 1F6 antibody
QVQLKESGPGLVQPSQTLSLTCTVSGFSLTSYTVSWVRQPPGKGLEWIAAISSGGSTYYNSALKSRLSISRDTSKSQVFLKMNSLQTEDTAMYFCARYTTDSGFDYWGQGVMVTVSS

SEQ ID NO: 108 - protein, 106 aa, synthetic construct
  REGION 1..106: note = VL sequence from 1F6 antibody
DIQMTQSPSVLSASVGDRVTLSCKASQNINKNLDWYQQKLGEAPKLLIYFTNNLQTGIPSRFSGSGSGTDYTLTISSLQPEDVATYYCYQYNSGWTFGGGTKLELK

SEQ ID NO: 109 - protein, 122 aa, synthetic construct
  REGION 1..122: note = VH sequence from 2H9 antibody
EVQLVESGGDLVQPGRSLKLSCASSGFTFSDYNMAWVRQAPKKGLEWVATISYDGTNTYYRDSVKGRFTISRDNAKSTLYLQMDSLRSEDTATYYCARQDSSYVYLSWFAYWGQGTLVTVSS

SEQ ID NO: 110 - protein, 107 aa, synthetic construct
  REGION 1..107: note = VL sequence from 2H9 antibody
DIQMTQSPASLSASLGETVTIECRASEDIYNGLTWYQQKPGKSPQLLISNANCLHTGVPSRFSGSGSGTQYSLKINSLQSEDVASYFCQQYYNYPWTFGGGTKLDLK

SEQ ID NO: 111 - protein, 117 aa, synthetic construct
  REGION 1..117: note = VH sequence from 7F10 antibody
QVQLKESGPGLVQPSQTLSLTCTVSGFSLTSYTVSWVRQPPGRGLEWIAAISSGGNTYYNSGLKSRLSISRDTSKSQVFLKMNSLQTEDTAMYFCARYAQIRGKDYWGQGVMVTVSS

SEQ ID NO: 112 - protein, 106 aa, synthetic construct
  REGION 1..106: note = VL sequence from 7F10 antibody
DIQMTQSPPILSASVGDRVTLSCKASQNINKNLDWYQQKHGEAPKLLIYYTHNLQTGIPSRFSGSGSGTDYTLTISSLQPEDVATYYCYQYYSGWTFGGGTKLQLK

SEQ ID NO: 113 - protein, 5 aa, synthetic construct
  REGION 1..5: note = VH CDR sequence, CDR1
SYTVS

SEQ ID NO: 114 - protein, 16 aa, synthetic construct
  REGION 1..16: note = VH CDR sequence, CDR2
AISSGGSTYYNSALKS

SEQ ID NO: 115 - protein, 9 aa, synthetic construct
  REGION 1..9: note = VH CDR sequence, CDR3
YTTDSGFDY

SEQ ID NO: 116 - protein, 11 aa, synthetic construct
  REGION 1..11: note = VL CDR sequence, CDR1
KASQNINKNLD

SEQ ID NO: 117 - protein, 7 aa, synthetic construct
  REGION 1..7: note = VL CDR sequence, CDR2
FTNNLQT

SEQ ID NO: 118 - protein, 8 aa, synthetic construct
  REGION 1..8: note = VL CDR sequence, CDR3
YQYNSGWT

SEQ ID NO: 119 - protein, 5 aa, synthetic construct
  REGION 1..5: note = VH CDR sequence, CDR1
DYNMA

SEQ ID NO: 120 - protein, 17 aa, synthetic construct
  REGION 1..17: note = VH CDR sequence, CDR2
TISYDGTNTYYRDSVKG

SEQ ID NO: 121 - protein, 13 aa, synthetic construct
  REGION 1..13: note = VH CDR sequence, CDR3
QDSSYVYLSWFAY

SEQ ID NO: 122 - protein, 11 aa, synthetic construct
  REGION 1..11: note = VL CDR sequence, CDR1
RASEDIYNGLT

SEQ ID NO: 123 - protein, 7 aa, synthetic construct
  REGION 1..7: note = VL CDR sequence, CDR2
NANCLHT

SEQ ID NO: 124 - protein, 9 aa, synthetic construct
  REGION 1..9: note = VL CDR sequence, CDR3
QQYYNYPWT

SEQ ID NO: 125 - protein, 5 aa, synthetic construct
  REGION 1..5: note = VH CDR sequence, CDR1
SYTVS

SEQ ID NO: 126 - protein, 16 aa, synthetic construct
  REGION 1..16: note = VH CDR sequence, CDR2
AISSGGNTYYNSGLKS

SEQ ID NO: 127 - protein, 9 aa, synthetic construct
  REGION 1..9: note = VH CDR sequence, CDR3
YAQIRGKDY

SEQ ID NO: 128 - protein, 11 aa, synthetic construct
  REGION 1..11: note = VL CDR sequence, CDR1
KASQNINKNLD

SEQ ID NO: 129 - protein, 7 aa, synthetic construct
  REGION 1..7: note = VL CDR sequence, CDR2
YTHNLQT

SEQ ID NO: 130 - protein, 8 aa, synthetic construct
  REGION 1..8: note = VL CDR sequence, CDR3
YQYYSGWT

SEQ ID NO: 131 - protein, 5 aa, synthetic construct
  REGION 1..5: note = glycine-serine linker
GGGGS

SEQ ID NO: 132 - protein, 5 aa, synthetic construct
  REGION 1..5: note = VH CDR sequence, CDR1
NYGMN

SEQ ID NO: 133 - protein, 16 aa, synthetic construct
  REGION 1..16: note = VH CDR sequence, CDR2
RIYPGSGSTNYQKFKG

SEQ ID NO: 134 - protein, 16 aa, synthetic construct
  REGION 1..16: note = VL CDR sequence, CDR1
RSSQSIVHSNGNTYLE

SEQ ID NO: 135 - protein, 7 aa, synthetic construct
  REGION 1..7: note = VL CDR sequence, CDR2
KVSNRPS

SEQ ID NO: 136 - protein, 9 aa, synthetic construct
  REGION 1..9: note = VL CDR sequence, CDR3
FQGSHVPWT

SEQ ID NO: 137 - protein, 5 aa, synthetic construct
  REGION 1..5: note = Linker
GGGGS

(19) United States
(12) Patent Application Publication
(10) Pub. No.: US 2025/0019434 A1
(43) Pub. Date: Jan. 16, 2025

(54) CHIMERIC ANTIGEN RECEPTOR

(71) Applicant: Autolus Limited, London (GB)

(72) Inventors: Martin Pulé, London (GB); Evangelia Kokalaki, London (GB); Shaun Cordoba, London (GB); Shimobi Onuoha, London (GB); Simon Thomas, London (GB); Biao Ma, London (GB); Mathieu Ferrari, London (GB)

(21) Appl. No.: 18/597,357

(22) Filed: Mar. 6, 2024

Related U.S. Application Data
(63) Continuation of application No. 17/054,670, filed on Nov. 11, 2020, now Pat. No. 11,963,981.

(30) Foreign Application Priority Data
May 15, 2018 (GB) 1807866.7
Jun. 14, 2018 (GB) 1809773.3

(51) Int. Cl. A61K 39/00; A61P 35/00; C07K 16/28

(52) U.S. Cl. CPC C07K 16/2803; A61K 39/4611; A61K 39/4631; A61K 39/464413; A61K 2239/28; A61P 35/00; C07K 2317/565; C07K 2317/622; C07K 2317/76

(57) ABSTRACT

The present invention provides a chimeric antigen receptor (CAR) which binds a target antigen having a bulky extra-cellular domain, wherein the CAR comprises a Fab antigen binding domain. The present invention also provides nucleic acid sequences and constructs encoding such a CAR, cells expressing such a CAR and their therapeutic uses.

### CROSS-REFERENCE TO RELATED APPLICATIONS

This application is a Continuation of U.S. application Ser. No. 17/054,670, filed Nov. 11, 2020, which is a U.S. National Phase of International Application No. PCT/GB2019/051330, filed May 15, 2019, which claims priority to Great Britain Application No. 1807866.7, filed May 15, 2018 and Great Britain Application No. 1809773.3, filed Jun. 14, 2018.

### INCORPORATION BY REFERENCE OF THE SEQUENCE LISTING

This application contains, as a separate part of disclosure, a Sequence Listing in computer-readable form (Filename: 55078B_SeqListing.xml; 180,284 bytes—XML file dated Feb. 21, 2024) which is incorporated by reference herein in its entirety.

### FIELD OF THE INVENTION

The present invention relates to a chimeric antigen receptor (CAR). In particular, it relates to a CAR having a Fab-like antigen binding domain.

### BACKGROUND TO THE INVENTION

### Chimeric Antigen Receptors (CARs)

A number of immunotherapeutic agents have been described for use in cancer treatment, including therapeutic monoclonal antibodies (mAbs), bi-specific T-cell engagers and chimeric antigen receptors (CARs).

Chimeric antigen receptors are proteins which graft the specificity of a monoclonal antibody (mAb) to the effector function of a T-cell. Their usual form is that of a type I transmembrane domain protein with an antigen recognizing amino terminus (binder), and a transmembrane domain connected to an endodomain which transmits T-cell activation signals.

The most common form of these molecules are fusions of single-chain variable fragments (scFv) derived from monoclonal antibodies, which recognize a target antigen, fused via a trans-membrane domain to a signalling endodomain. Such molecules result in activation of the T-cell in response to recognition by the scFv of its target. When T cells express such a CAR, they recognize and kill target cells that express the target antigen. CARs have been developed against various tumour-associated antigens and many are currently undergoing clinical trials.

Although CAR-T cell-mediated treatment have shown success towards compact target antigens such as CD19 or GD2, chimeric antigen receptors often to fail to signal in response to antigens with bulky extracellular domains.

An optimum synaptic distance is required for efficient triggering of downstream signalling after antigen encounter. Upon T cell encounter with an antigen presenting cell (via TCR interaction with peptide MHC), proteins at the interface segregate passively based on size. Phosphatases such as CD45 and CD148, which have large ectodomains, are excluded from regions of close contact between the T cell and APC (see FIG. 1). The synapse formed through interaction of peptide MHC and TCR is optimal for occlusion of CD45. In the case of CAR-T cells targeting smaller antigens such as CD19, there is no barrier to synapse formation and such antigens can be targeted efficiently at multiple epitopes. Large proteins such as CD22 pose a unique problem, as illustrated in FIG. 2. Targeting a membrane distal epitope on such proteins is likely to provide a suboptimal synapse length allowing phosphatases to enter the synapse and inhibit tyrosine phosphorylation. Targeting membrane proximal regions may improve synapse formation, however steric occlusion of the epitope is likely to lead to suboptimal ligation of the target allowing the presence of phosphatases within the synapse, dampening tyrosine phosphorylation, kinase activity and thus CAR signaling.

There is therefore a need for alternative CAR T-cell approaches, capable of killing target cells expressing a large or bulky target antigen.

### DESCRIPTION OF THE FIGURES

FIG. 1—Schematic diagram illustrating the relative sizes of the extracellular domains of a Fab CAR (Fab), a classical CAR having an scFv antigen binding domain (scFv), the phosphatase CD45, a CD4/TCR/MHC complex and a CD2:CD58 complex. When a T cell interacts with a tumour cell, either via a TCR:MHC interaction or a CAR:target antigen interaction, an immunological synapse is formed and phosphatases such as CD45 and CD148 are excluded.

FIG. 2—Schematic diagram illustrating the relative sizes of the extracellular domains of CD22, CD45, a CD4/TCR/MHC complex and a CD2:CD58 complex. CD22 has a very large, bulky extracellular domain. This makes it a difficult target for a CAR T-cell as the combined length of the CAR extracellular domain and the CD22 extracellular domain is too long for an optimal T-cell;Target cell synapse, meaning that phosphatases such as CD45 and CD148 are not efficiently excluded.

FIG. 3—Schematic diagram illustrating the T-cell: target cell synapse for (A) a FabCAR targeting CD22, and b) a classical scFv CAR targeting CD22. Surprisingly, although the extracellular domain is longer and more bulky than an scFv CAR extracellular domain and therefore might be predicted to compound the issues related to the target antigen having a large and bulky extracellular domain, the FabCAR results in better CAR-mediated signalling upon target cell encounter, and more efficient killing of target cells.

FIG. 4—Graph comparing killing of CD22-expressing target cells by T cells expressing a FabCAR or an scFv CAR. T cells were transduced with viral vectors expressing either a Fab CAR or an scFv CAR having a CD8 stalk spacer. The antigen binding domains were based on the same anti-CD22 antibody, either 10C1 or 1D9-3. T cells were co-cultured with CD22-expressing SupT1 target cells for 24 hours and absolute number of target cells was calculated, and the number in the CAR normalised according to the target number in the non-transduced (NT) condition. The normalised data are expressed as a percentage of target cell survival.

FIG. 5—Histograms showing T-cell proliferation after 4 days co-culture with target cells. CD56-depeleted CAR expressing T cells were co-cultured with Raji target cells and analysed by flow cytometry to measure the dilution of the Cell Trace Violet (CTV) which occurs as the T-cells divide. The T cells labelled with CTV are excited with a 405 nm (violet) laser. The same panel of constructs were tested as for the killing assay, namely: a 10C1 FabCAR; a 10C1 scFv CAR having a CD8 stalk spacer; a 1D9-3 FabCAR and a 1D9-3 scFv CAR having a CD8 stalk spacer.

FIG. 6—Different binding domain formats of chimeric antigen receptors
(a) Fab CAR format; (b) dAb CAR format; (c) scFv CAR format

FIG. 7—CAR OR gate targeting CD19, CD22 and CD79 using different format CARs
(a) A tricistronic cassette can be generated be separating the coding sequences for the two receptors using two FMD-2A sequences; (b) OR gate combining three different formats: scFv-CAR for CD19, Fab CAR for CD22 and dAb CAR for CD79

FIG. 8—Graph showing the results of a FACS-based killing assay comparing target cell killing by T cells expressing a FabCAR having a 9A8 antigen-binding domain and T cells expressing a FabCAR having a 3B4 antigen-binding domain

FIG. 9—Graph showing the IL-2 release following 72 hours co-culture with SupT1 target cells comparing T cells expressing a FabCAR having a 9A8 antigen-binding domain and T cells expressing a FabCAR having a 3B4 antigen-binding domain

FIG. 10—Graph showing the results of a FACS-based killing assay comparing target cell killing by T cells expressing various anti-CD22 FabCARs. Target cells were either non-transduced SupT1 cells (A); or SupT1 cells transduced to express CD22, showing one of three levels of CD22 expression:
B—“ultra-low” undetectable by flow cytometry
C—“low” averaging 255 copies of CD22 per cell
D—“high” averaging 78,916 copies of CD22 per cell

FIG. 11—Schematic diagram illustrating the problem of cross-pairing when expressing two FabCARs.

FIG. 12—Schematic diagram illustrating “Crossmab” and “Ortho-Fab” to avoid cross-pairing between FabCARs.

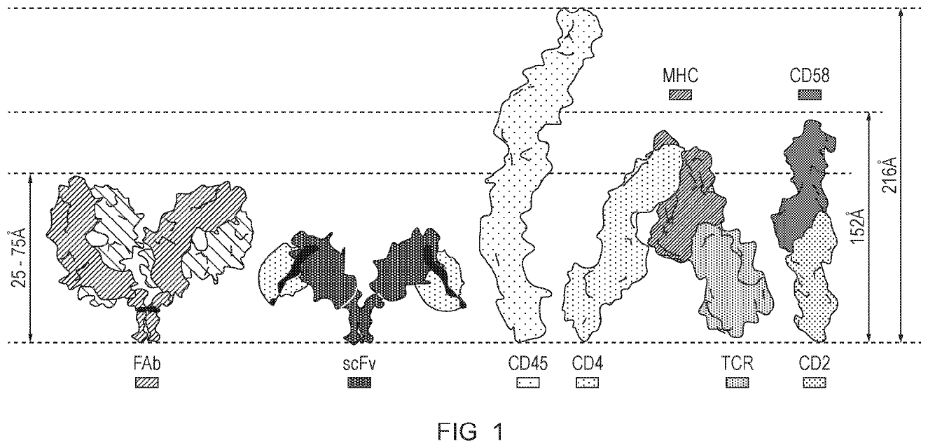

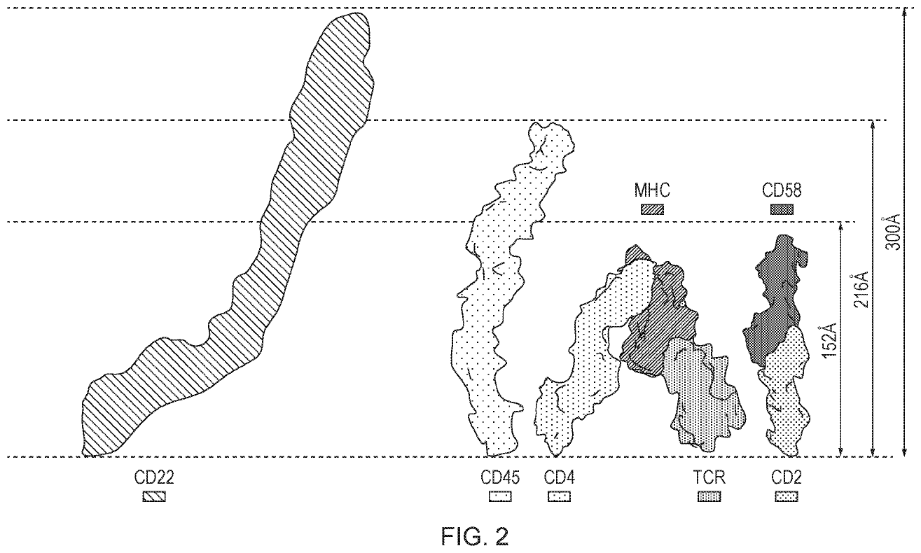

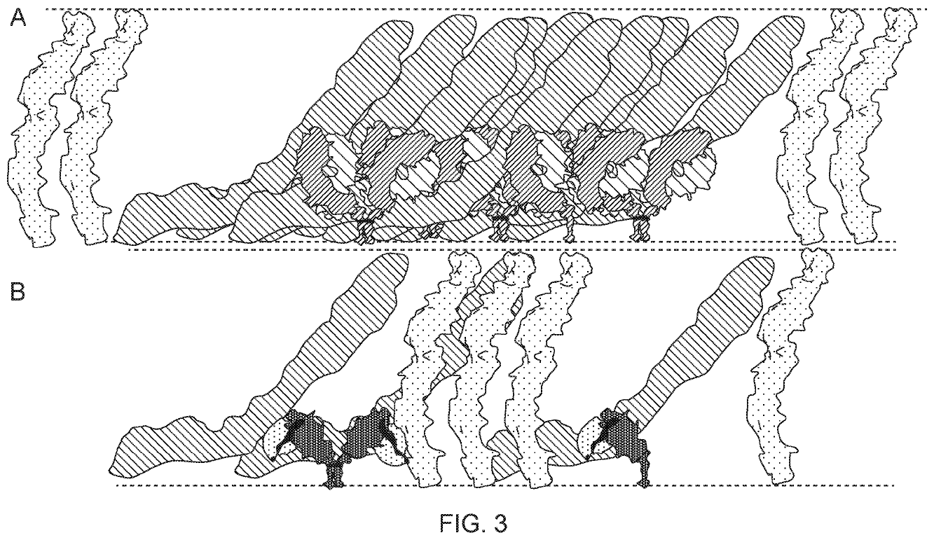

### SUMMARY OF ASPECTS OF THE INVENTION

The present inventors have found that it is possible to improve CAR-mediated targeting of bulky antigens and the efficiency of CAR-mediated killing of target cells expressing bulky target antigens, using a CAR having a Fab binding domain as opposed to an scFv binding domain.

Thus, in a first aspect the present invention provides a chimeric antigen receptor (CAR) which binds a target antigen having a bulky extracellular domain, wherein the CAR comprises a Fab antigen binding domain.

The target antigen may have an extracellular domain of at least about 150 Å.

The target antigen may have an extracellular domain of at least about 400 amino acids.

The target antigen may be selected from the following group: CD22, CD21, CEACAM5, MUC1 or FcRL5. In particular, the target antigen may be CD22.

The antigen-binding domain may comprise;
a) a heavy chain variable region (VH) having complementarity determining regions (CDRs) with the following sequences:

CDR1
(SEQ ID No. 93)
NFAMA
CDR2
(SEQ ID No. 94)
SISTGGGNTYYRDSVKG
CDR3
(SEQ ID No. 95)
QRNYYDGSYDYEGYTMDA;

and
b) a light chain variable region (VL) having complementarity determining regions (CDRs) with the following sequences:

CDR1
(SEQ ID No. 96)
RSSQDIGNYLT
CDR2
(SEQ ID No. 97)
GAIKLED
CDR3
(SEQ ID No. 98)
LQSIQYP.

The antigen-binding domain may comprise:
a) a heavy chain variable region (VH) having complementarity determining regions (CDRs) with the following sequences:

CDR1
(SEQ ID No. 101)
TSGMGVG
CDR2
(SEQ ID No. 102)
NIWWDDDKNYNPSLKN
CDR3
(SEQ ID No. 103)
IAHYFDGYYYVMDV;

and
b) a light chain variable region (VL) having complementarity determining regions (CDRs) with the following sequences:

CDR1
(SEQ ID No. 104)
LASGGISNDLA
CDR2
(SEQ ID No. 105)
AASRLQD
CDR3
(SEQ ID No. 106)
QQSYKYPY

The CAR may comprise a VH domain having the sequence shown as SEQ ID No. 65; and a VL domain having the sequence shown as SEQ ID No. 66.

The CAR may comprise a VH domain having the sequence shown as SEQ ID No. 99; and a VL domain having the sequence shown as SEQ ID No. 100.

In a second aspect, there is provided a nucleic acid sequence which encodes a CAR according to the first aspect of the invention.

The nucleic acid sequence may have the following general structure:

VH-CH-spacer-TM-endo-coexpr-VL-CL
in which:
VH is a nucleic acid sequence encoding a heavy chain variable domain of a first polypeptide;
CH is a nucleic acid sequence encoding a heavy chain constant domain of the first polypeptide;
spacer is a nucleic acid sequence encoding a spacer of the first polypeptide;
TM is a nucleic acid sequence encoding a transmembrane region of the first polypeptide;
endo is a nucleic acid sequence encoding an endodomain of the first polypeptide;
VL is a nucleic acid sequence encoding a light chain variable domain of a second polypeptide;
CL is a nucleic acid sequence encoding a light chain constant domain of the second polypeptide; and
coexpr is a nucleic acid sequence enabling the co-expression of the first and second polypeptides.

In a third aspect, there is provided a nucleic acid construct which comprises a first nucleic acid sequence according to the second aspect of the invention, and a second nucleic acid sequence encoding a second chimeric antigen receptor which has a domain antibody (dAb) or scFv antigen binding domain.

In particular, there is provided a nucleic acid construct which comprises a first nucleic acid sequence according to the second aspect of the invention; a second nucleic acid sequence encoding a second chimeric antigen receptor which has a domain antibody (dAb) antigen binding domain; and a third nucleic acid sequence encoding a third CAR which has an scFv antigen binding domain.

The first nucleic acid sequence may encode an anti-CD22 Fab CAR; the second nucleic acid sequence may encode an anti-CD79 dAb CAR; and the third nucleic acid sequence may encode an anti-CD19 scFv CAR.

In a fourth aspect, there is provided a vector which comprises a nucleic acid sequence according to the second aspect of the invention or a nucleic acid construct according to the third aspect of the invention.

In a fifth aspect, there is provided a cell which expresses a CAR according to the second aspect of the invention.

In particular, there is provided a cell which expresses a first CAR according to the first aspect of the invention, and a second chimeric antigen receptor which has a domain antibody (dAb) or scFv antigen binding domain.

In particular, there is provided a cell which expresses a first CAR according to the first aspect of the invention; a second CAR which has a domain antibody (dAb) antigen binding domain; and a third CAR which has an scFv antigen binding domain.

The first CAR may be an anti-CD22 Fab CAR; the second CAR may be an anti-CD79 dAb CAR; and the third CAR may be an anti-CD19 scFv CAR.

In a sixth aspect, there is provided a method for making a cell according to the fifth aspect of the invention, which comprises the step of introducing a nucleic acid sequence according to the second aspect of the invention; a nucleic acid construct according to the third aspect of the invention; or a vector according to the fourth aspect of the invention into a cell ex vivo.

In a seventh aspect, there is provided a pharmaceutical composition which comprises a plurality of cells according the fifth aspect of the invention, together with a pharmaceutically acceptable carrier, diluent or excipient.

In an eighth aspect, there is provided a method for treating cancer which comprises the step of administering a pharmaceutical composition according to the seventh aspect of the invention to a subject.

The cancer may, for example, be a B-cell lymphoma or leukemia.

In a ninth aspect, there is provided a pharmaceutical composition according to the seventh aspect of the invention for use in treating cancer.

In a tenth aspect, there is provided the use of a cell according to the fifth aspect of the invention in the manufacture of a pharmaceutical composition for treating cancer.

The present invention provides chimeric antigen receptors which show improved CAR-mediated signalling and target cell killing when targeting antigens with bulky extracellular domains. Such antigens are difficult to target with a classical CAR as they form sub-optimal T-cell: target cell synapses.

To capacity to target such antigens opens up whole new possibilities for cancer treatment. Many potentially useful cancer target antigens have bulky extracellular domains, for example, CD22, CD21, CEACAM5, MUC1 or FcRL5. The present invention provides improved constructs for targeting these antigens, enabling them to be used as single targets and, importantly, to be included in strategies for targeting multiple antigens in order to increase CAR-T cell efficacy and safety.

### DETAILED DESCRIPTION

### Chimeric Antigen Receptors

The present invention relates to a chimeric antigen receptor with a Fab antigen-binding domain.

A classical chimeric antigen receptor (CAR) is a chimeric type I trans-membrane protein which connects an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8α and even just the IgG1 hinge alone, depending on the antigen. A trans-membrane domain anchors the protein in the cell membrane and connects the spacer to the endodomain.

Early CAR designs had endodomains derived from the intracellular parts of either the γ chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3ζ results in second generation receptors which can transmit an activating and co-stimulatory signal simultaneously after antigen recognition. The co-stimulatory domain most commonly used is that of CD28. This supplies the most potent co-stimulatory signal-namely immunological signal 2, which triggers T-cell proliferation. Some receptors have also been described which include TNF receptor family endodomains, such as the closely related OX40 and 41BB which transmit survival signals. Even more potent third generation CARs have now been described which have endodomains capable of transmitting activation, proliferation and survival signals.

When the CAR binds the target-antigen, this results in the transmission of an activating signal to the T-cell it is expressed on. Thus the CAR directs the specificity and cytotoxicity of the T cell towards tumour cells expressing the targeted antigen.

CARs typically therefore comprise: (i) an antigen-binding domain; (ii) a spacer; (iii) a transmembrane domain; and (iii) an intracellular domain which comprises or associates with a signalling domain.

A CAR may have the general structure:

Antigen binding domain-spacer domain-transmembrane domain-intracellular signaling domain (endodomain).

### Antigen Binding Domain

The antigen binding domain is the portion of the chimeric receptor which recognizes antigen. In a classical CAR, the antigen-binding domain comprises: a single-chain variable fragment (scFv) derived from a monoclonal antibody (see FIG. 6c). CARs have also been produced with domain antibody (dAb) or VHH antigen binding domains (see FIG. 6b).

In the chimeric antigen receptors of the present invention, the antigen binding comprises a Fab fragment of, for example, a monoclonal antibody (see FIG. 6a). A FabCAR comprises two chains: one having an antibody-like light chain variable region (VL) and constant region (CL); and one having a heavy chain variable region (VH) and constant region (CH). One chain also comprises a transmembrane domain and an intracellular signalling domain. Association between the CL and CH causes assembly of the receptor.

The two chains of a Fab CAR may have the general structure:

VH-CH-spacer-transmembrane domain-intracellular signalling domain; and VL-CL

or

VL-CL-spacer-transmembrane domain-intracellular signalling domain; and VH-CH

For the Fab-type chimeric receptors described herein, the antigen binding domain is made up of a VH from one polypeptide chain and a VL from another polypeptide chain.

The polypeptide chains may comprise a linker between the VH/VL domain and the CH/CL domains. The linker may be flexible and serve to spatially separate the VH/VL domain from the CH/CL domain.

Flexible linkers may be composed of small, non-polar residues such as glycine, threonine and serine. The linker may comprise one or more repeats of a glycine-serine linker, such as a (Gly₄Ser)ₙ linker, where n is the number of repeats. The or each linker may be less than 50, 40, 30, 20 or 10 amino acids in length.

### Constant Region Domains

There are two types of light chain in humans: kappa (κ) chain and lambda (λ) chain. The lambda class has 4 subtypes: λ₁, λ₂, λ₃ and λ₄. The light chain constant region of a Fab-type chimeric receptor may be derived from any of these light chain types.

The light chain constant domain of a chimeric receptor of the present invention may have the sequence shown as SEQ ID NO. 1 which is a kappa chain constant domain.

SEQ ID No. 1
TVAAPSVFIFPPSDEQLKSGTASVVCLLNNFYPREAKVQWKVDN
ALQSGNSQESVTEQDSKDSTYSLSSTLTLSKADYEKHKVYACEV
THQGLSSPVTKSFNRGEC

There are five types of mammalian immunoglobulin heavy chain: γ, δ, α, μ and ε which define the classes of immunoglobulin IgG, IgD, IgA, IgM and IgE respectively. Heavy chains γ, δ and α have a constant domain composed of three tandem Ig domain and have a hinge for added flexibility. Heavy chains μ and ε are composed of four domains.

The CH domain of a Fab-type chimeric receptor of the present invention may comprise the sequence shown as SEQ ID No. 2 which is from a γ immunoglobulin heavy chain.

SEQ ID No. 2
STKGPSVFPLAPSSKSTSGGTAALGCLVKDYFPEPVTVSWNSGA
LTSGVHTFPAVLQSSGLYSLSSVVTVPSSSLGTQTYICNVNHKP
SNTKVDKRV

### Spacer

Classical CARs comprise a spacer sequence to connect the antigen-binding domain with the transmembrane domain and spatially separate the antigen-binding domain from the endodomain. A flexible spacer allows the antigen-binding domain to orient in different directions to facilitate binding.

In a FabCAR (FIG. 6A), as in a classical chimeric antigen receptor (FIG. 6C) and a dAb CAR (FIG. 6B), the spacer may cause two of the polypeptide chains to dimerise. Two of the polypeptide chains may, for example, comprise one or more suitable cysteine residues to form di-sulphide bridge(s). The hinge from IgG1 is suitable in this regard. A spacer based on an IgG1 hinge may have the sequence shown as SEQ ID. No. 3

(human IgG1 hinge):
SEQ ID No. 3
AEPKSPDKTHTCPPCPKDPK

Alternatively, a hinge spacer may have the sequence shown as SEQ ID No. 4

(hinge spacer)
SEQ ID No. 4
EPKSCDKTHTCPPCP

In the FabCAR of the invention, the two poypeptides of a dimeric FabCAR, as illustrated in FIG. 6A are identical. They have the same antigen binding domains derived from the same antibody, and they bind the same epitope on the same target antigen. The first and second polypeptides in the dimer are simply duplicate copies of a polypeptide encoded from the same transcript.

### Transmembrane Domain

The transmembrane domain is the portion of the chimeric receptor which spans the membrane. The transmembrane domain may be any protein structure which is thermodynamically stable in a membrane. This is typically an alpha helix comprising of several hydrophobic residues. The transmembrane domain of any transmembrane protein can be used to supply the transmembrane portion of the chimeric receptor. The presence and span of a transmembrane domain of a protein can be determined by those skilled in the art using the TMHMM algorithm (http://www.cbs.dtu.dk/services/TMHMM-2.0/). Alternatively, an artificially designed TM domain may be used.

### Endodomain

The endodomain is the signal-transmission portion of the chimeric receptor. It may be part of or associate with the intracellular domain of the chimeric receptor. After antigen recognition, receptors cluster, native CD45 and CD148 are excluded from the synapse and a signal is transmitted to the cell. The most commonly used endodomain component is that of CD3-zeta which contains 3 ITAMs. This transmits an activation signal to the T cell after antigen is bound. CD3-zeta may not provide a fully competent activation signal and additional co-stimulatory signalling may be needed. Co-stimulatory signals promote T-cell proliferation and survival. There are two main types of co-stimulatory signals: those that belong the Ig family (CD28, ICOS) and the TNF family (OX40, 41BB, CD27, GITR etc). For example, chimeric CD28 and OX40 can be used with CD3-Zeta to transmit a proliferative/survival signal, or all three can be used together.

The endodomain may comprise:
(i) an ITAM-containing endodomain, such as the endodomain from CD3 zeta; and/or
(ii) a co-stimulatory domain, such as the endodomain from CD28 or ICOS; and/or
(iii) a domain which transmits a survival signal, for example a TNF receptor family endodomain such as OX-40, 4-1BB, CD27 or GITR.

A number of systems have been described in which the antigen recognition portion is on a separate molecule from the signal transmission portion, such as those described in WO015/150771; WO2016/124930 and WO2016/030691. The chimeric receptor of the present invention may therefore comprise an antigen-binding component comprising an antigen-binding domain and a transmembrane domain; which is capable of interacting with a separate intracellular signalling component comprising a signalling domain. The vector of the invention may express a chimeric receptor signalling system comprising such an antigen-binding component and intracellular signalling component.

The chimeric receptor may comprise a signal peptide so that when it is expressed inside a cell, the nascent protein is directed to the endoplasmic reticulum and subsequently to the cell surface, where it is expressed. The signal peptide may be at the amino terminus of the molecule.

### Target Antigen

A ‘target antigen’ is an entity which is specifically recognised and bound by the antigen-binding domains of a chimeric receptor of the invention.

The target antigen may be an antigen present on a cancer cell, for example a tumour-associated antigen.

The target antigen may have a relatively long and/or bulky extracellular domain. The extracellular domain of CD45 is 216 Å in size. Depending on the spacer used typically the antigen binding domain of a classical CAR will measure in the range of 25-75 Å, as such, antigens larger than 150 Å are difficult to target due to the poor synapse formation, leading to the presence of phosphatases within said synapse.

The target antigen may have an extracellular domain which is greater than about 150 Å, for example the target antigen may have an extracellular domain which is 150-400 Å, 200-350 Å or 250-310 Å in size.

There is a correlation between the size of the molecule and the amino acid length of the extracellular domain of target antigens. Examples of the size of the extracellular domain and the number of amino acids is shown in the table below both for antigens with compact extracellular domains (EpCAM, CD19) and antigens with bulky extracellular domains (CEACAM5, CD22).

| Protein | Extracellular domain size | | Amino acids |
|---|---|---|---|
| EpCAM | 48 | Å | 242 |
| CD19 | 63 | Å | 272 |
| CEACAM5 | 280 | Å | 651 |
| CD22 | 306 | Å | 668 |

The target antigen may have an extracellular domain which is greater than about 400 amino acids in length, for example the target antigen may have an extracellular domain which is 400-1000, 500-900, 600-800 or 600-700 amino acids in length.

The extracellular domain of CD22 has seven IgG-like domains in its extracellular domain. The target antigen of the chimeric receptor of the invention may have a length equivalent to at least 4, 5, 6 or 7 Ig-like domains. The extracellular domain of CD21 has 21 short consensus repeats (SCR) of about 60 amino acids each. The target antigen of the chimeric receptor of the invention may have a length equivalent to at least 15, 17, 19 or 21 CSRs.

The target antigen may have an extracellular domain which is longer than the optimal intracellular distance between a T-cell and a target cell at a T-cell: target cell synapse. The target cell may have an extracellular domain which is at least 40, 50, 60 or 70 nM

The target antigen may be CD22, CD21, CEACAM5, MUC1 or FCRL5

### CD22

CD22 has seven extracellular IgG-like domains, which are commonly identified as Ig domain 1 to Ig domain 7, with Ig domain 7 being most proximal to the B cell membrane and Ig domain 1 being the most distal from the Ig cell membrane.

The positions of the Ig domains in terms of the amino acid sequence of CD22 (http://www.uniprot.org/uniprot/P20273) are summarised in the following table:

| Ig domain | Amino acids |
|---|---|
| 7 | 20-138 |
| 6 | 143-235 |
| 5 | 242-326 |
| 4 | 331-416 |
| 3 | 419-500 |
| 2 | 505-582 |
| 1 | 593-676 |

Examples of anti-CD22 CARs with antigen-binding domains derived from m971, HA22 and BL22 scFvs are described by Haso et al. (Blood; 2013; 121(7)). The antibodies HA22 and BL22 bind to an epitope on Ig domain 5 of CD22.

Other anti-CD22 antibodies are known, such as the mouse anti-human CD22 antibodies 1D9-3, 3B4-13, 7G6-6, 6C4-6, 4D9-12, 5H4-9, 10C1-D9, 15G7-2, 2B12-8, 2C4-4 and 3E10-7; and the humanised anti-human CD22 antibodies LT22 and Inotuzumab (G5_44). Table 1 summarises the, VH, VL and CDR sequences (in bold and underlined) and the position of the target epitope on CD22 for each antibody.

TABLE 1

| | | | Position |
|---|---|---|---|
| | | | of |
| | | | epitope |
| Antibody | VH | VL | on CD22 |
| 1D9-3 | EVQLVESGGGLVQPKGSLK | DIVMTQSQKFMSTSVGD | Domain 1 |
| | LSCAASGFTFNTYAMHWVR | RVSITCKASQNVRTAVA | and 2 |
| | QAPGKGLEWVARIRSKSSN | WYQQKPGQSPKALIYLA | |
| | YATYYADSVKDRFTISRDD | SNRHTGVPDRFTGSGSG | |
| | SQSMLYLQMNNLKTEDTAM | TDFTLTISNVQSEDLADY | |
| | YYCVVDYLYAMDYWGQGT | FCLQHWNYPFTFGSGTK | |
| | SVTVSS | LEIK | |
| | (SEQ ID No. 39) | (SEQ ID No. 40) | |
| 3B4-13 | QVQLQQSGAELVRPGASVT | QAVVTQESALTTSPGET | Domain 1 |
| | LSCKASGYTFTDYEMHWVK | VTLTCRSSAGAVTTSNY | and 2 |
| | QTPVHGLEWIGAIDPETGA | ANWVQEKPDHLFTGLIG | |
| | TAYNQKFKGKAILTADKSSS | GTNNRAPGVPARFSGSL | |
| | TAYMDLRSLTSEDSAVYYC | IGDKAALTITGAQTEDEAI | |
| | TRYDYGSSPWFAYWGQGT | YFCALWNSNHWVFGGG | |
| | LVTVSA | TKLTVL | |
| | (SEQ ID No. 41) | (SEQ ID No. 42) | |
| 7G6-6 | QVQLQQPGAELVMPGASV | DIVMSQSPSSLAVSVGE | Domain 1 |
| | KLSCKASGYTFTSYWMHW | KVTMSCKSSQSLLYSSN | and 2 |
| | VKQRPGQGLEWIGEIDPSD | QKNYLAWYQQKPGQSP | |
| | SYTNYNQKFKGKATLTVDK | KLLIYWASTRESGVPDRF | |
| | SSSTAYMQLSSLTSEDSAV | TGSGSGTDFTLTISSVKA | |
| | YYCARGYYGSSSFDYWGQ | EDLAVYYCQQYYSYTFG | |
| | GTTLTVSS | GGTKLEIK | |
| | (SEQ ID No. 43) | (SEQ ID No. 44) | |
| 6C4-6 | QVQLKESGPGLVAPSQSLSI | DIQMTQSPASLSASVGE | Domain 3 |
| | TCTVSGFSLTSYGVHWVRQ | TVTITCRASENIYSYLAW | |
| | PPGKGLEWLVVIWSDGSTT | YQQKQGKSPQLLVYNAK | |
| | YNSALKSRLSISKDNSKSQ | TLAEGVPSRFSGSGSGT | |
| | VFLKMNSLQTDDTAMYYCA | QFSLKINSLQPEDFGSYY | |
| | RHADDYGFAWFAYWGQG | CQHHYGTPPTFGGGTKL | |
| | TLVTVSA | EIK | |
| | (SEQ ID No. 45) | (SEQ ID No. 46) | |
| 4D9-12 | EFQLQQSGPELVKPGASVK | DIQMTQSPSSLSASLGE | Domain 4 |
| | ISCKASGYSFTDYNMNWVK | RVSLTCRASQEISGYLS | |
| | QSNGKSLEWIGVINPNYGT | WLQQKPDGTIKRLIYAAS | |
| | TSYNQKFKGKATLTVDQSS | TLDSGVPKRFSGSRSGS | |
| | STAYMQLNSLTSEDSAVYY | DYSLTISSLESEDFADYY | |
| | CARSSTTVVDWYFDVWGT | CLQYASYPFTFGSGTKL | |
| | GTTVTVSS | EIK | |
| | (SEQ ID No. 47) | (SEQ ID No. 48) | |
| 5H4-9 | QVQVQQPGAELVRPGTSV | DVVMTQTPLSLPVSLGD | Domain 4 |
| | KLSCKASGYTFTRYWMYW | QASISCRSSQSLVHSNG | |
| | VKQRPGQGLEWIGVIDPSD | NTYLHWYLQKPGQSPKL | |
| | NFTYYNQKFKGKATLTVDT | LIYKVSNRFSGVPDRFSG | |
| | SSSTAYMQLSSLTSEDSAV | SGSGTDFTLKISRVEAED | |
| | YYCARGYGSSYVGYWGQG | LGVYFCSQSTHVPPWTF | |
| | TTLTVSS | GGGTKLEIK | |
| | (SEQ ID No. 49) | (SEQ ID No. 50) | |
| 10C1-D9 | QVTLKESGPGILQSSQTLSL | DIQMTQTTSSLSASLGDR | Domain 4 |
| | TCSFSGFSLSTSDMGVSWI | VTISCRASQDISNYLNWY | |
| | RQPSGKGLEWLAHIYWDD | QQKPDGTVKLLIYYTSRL | |
| | DKRYNPSLKSRLTISKDASR | HSGVPSRFSGSGSGTDY | |
| | NQVFLKIATVDTADTATYYC | SLTISNLEQEDIATYFCQ | |
| | ARSPWIYYGHYWCFDVWG | QGNTLPFTFGSGTKLEIK | |
| | TGTTVTVSS | (SEQ ID No. 52) | |
| | (SEQ ID No. 51) | | |
| 15G7-2 | QVQLQQSGAELVKPGASVK | QIVLTQSPAIMSASPGEK | Domain 4 |
| | LSCKASGYTFTEYTIHWVK | VTMTCSASSSVSYMYW | |
| | QRSGQGLEWIGWFYPGSG | YQQKPGSSPRLLIYDTSN | |
| | SIKYNEKFKDKATLTADKSS | LASGVPVRFSGSGSGTS | |
| | STVYMELSRLTSEDSAVYF | YSLTISRMEAEDAATYYC | |
| | CARHGDGYYLPPYYFDYW | QQWSSYPLTFGAGTKLE | |
| | GQGTTLTVSS | LK | |
| | (SEQ ID No. 53) | (SEQ ID No. 54) | |
| 2B12-8 | QVQLQQSGAELARPGASVK | DIVLTQSPATLSVTPGDS | Domain 4 |
| | LSCKASGYIFTSYGISWVKQ | VSLSCRASQSISTNLHW | |
| | RTGQGLEWIGEIYPRSGNT | YQQKSHASPRLLIKYASQ | |
| | YYNEKFKGKATLTADKSSS | SVSGIPSRFSGSGSGTD | |
| | TAYMELRSLTSEDSAVYFC | FTLSINSVETEDFGIFFCQ | |
| | ARPIYYGSREGFDYWGQGT | QSYSWPYTFGGGTKLEI | |
| | TLTVSS | K | |
| | (SEQ ID No. 55) | (SEQ ID No. 56) | |
| 2C4-4 | QVQLQQPGAELVMPGASV | DVLMTQTPLSLPVSLGD | Domain 5- |
| | KLSCKASGYTFTSYWMHW | QASISCRSSQSIVHSNGN | 7 |
| | VKQRPGQGLEWIGEIDPSD | TYLEWYLQKPGQSPKLLI | |
| | SYTNYNQKFKGKSTLTVDK | YKVSNRFSGVPDRFSGS | |
| | SSSTAYIQLSSLTSEDSAVY | ESGTDFTLKISRVEAEDL | |
| | YCARWASYRGYAMDYWG | GVYYCFQGSHVPWTFG | |
| | QGTSVTVSS | GGTKLEIK | |
| | (SEQ ID No. 57) | (SEQ ID No. 58) | |
| 3E10-7 | EFQLQQSGPELVKPGASVK | DIQMTQSPSSLSASLGE | Domain 5- |
| | ISCKASGYSFTDYNMNWVK | RVSLTCRASQEISGYLS | 7 |
| | QSNGKSLEWIGVINPNYGT | WLQQKPDGTIKRLIYAAS | |
| | TSYNQRFKGKATLTVDQSS | TLDSGVPKRFSGSRSGS | |
| | STAYMQLNSLTSEDSAVYY | DYSLTISSLESEDFADYY | |
| | CARSGLRYWYFDVWGTGT | CLQYASYPFTFGSGTKL | |
| | TVTVSS | EIK | |
| | (SEQ ID No. 59) | (SEQ ID No. 60) | |
| LT22 | EVQLVESGAEVKKPGSSVK | DIVMTQSPATLSVSPGER | Domain 5 |
| | VSCKASGYTFTNYWINWVR | ATLSCRSSQSLVHSNGN | |
| | QAPGQGLEWMGNIYPSDS | TYLHWVYQQKPGQAPRLL | |
| | FTNYNQKFKDRVTITADKST | IYKVSNRFSGVPARFSG | |
| | STVYLELRNLRSDDTAVYY | SGSGAEFTLTISSLQSED | |
| | CTRDTQERSWYFDVWGQG | FAVYYCSQSTHVPWTFG | |
| | TLVTVSS | QGTRLEIKR | |
| | (SEQ ID No. 61) | (SEQ ID No. 62) | |
| Inotuzumab | EVQLVQSGAEVKKPGASVK | DVQVTQSPSSLSASVGD | Domain 7 |
| G5_44 | VSCKASGYRFTNYWIHWVR | RVTITCRSSQSLANSYG | |
| | QAPGQGLEWIGGINPGNNY | NTFLSWYLHKPGKAPQL | |
| | ATYRRKFQGRVTMTADTST | LIYGISNRFSGVPDRFSG | |
| | STVYMELSSLRSEDTAVYY | SGSGTDFTLTISSLQPED | |
| | CTREGYGNYGAWFAYWG | FATYYCLQGTHQPYTFG | |
| | QGTLVTVSS | QGTKVEIKR | |
| | (SEQ ID No. 63) | (SEQ ID No. 64) | |
| 9A8-1 | EVQLVESGGGLVQPGRSLK | DIQMTQSPSSLSASLGD | Domains |
| | LSCAASGFTFSNFAMAWVR | RVTITCRSSQDIGNYLTW | 1 and 2 |
| | QPPTKGLEWVASISTGGGN | FQQKVGRSPRRMIYGAI | |
| | TYYRDSVKGRFTISRDDAK | KLEDGVPSRFSGSRSGS | |
| | NTQYLQMDSLRSEDTATYY | DYSLTISSLESEDVADYQ | |
| | CARQRNYYDGSYDYEGYT | CLQSIQYPFTFGSGTKLE | |
| | MDAWGQGTSVTVSS (SEQ | IK (SEQ ID No. 66) | |
| | ID No. 65) | | |
| 1G3-4 | QVTLKESGPGILQPSQTLSL | DIQMTQSPASLSASLGET | Domain 4 |
| | TCTFSGFSLSTSGMGVGWI | VSIECLASGGISNDLAWY | |
| | RQPSGKGLEWLTNIWWDD | QQKSGKSPQLLIYAASR | |
| | DKNYNPSLKNRLTISKDTSI | LQDGVPSRFSGSGSGTR | |
| | NQAFLKITNVDTADTATYYC | YSLKISGMQSEDEADYF | |
| | ARIAHYFDGYYYVMDVWG | CQQSYKYPYTFGGGTKL | |
| | QGTSVTVSS (SEQ ID No. | ELK (SEQ ID No. | |
| | 99) | 100) | |

An antigen binding domain of a FabCAR which binds to CD22 may comprise the VH and/or VL sequence from any of the CD22 antibodies listed in table 1, or a variant thereof which has at least 70, 80, 90 or 90% sequence identity, which variant retains the capacity to bind CD22.

### CD21

CD21, also known as CR2 is a protein expressed on mature B cells and follicular dendritic cells which is involved in the complement system. On mature B cells, CD21 forms the B cell coreceptor complex with CD19 and CD81. When membrane IgM binds to the antigen, CD21 binds to antigens through the attached C3d.

Mature CD21 is 1,408 amino acids that includes 21 short consensus repeats (SCR) of about 60 amino acids each, plus transmembrane and cytoplasmic regions.

Commercially available monoclonal antibodies against CD21 are known, such as MAB4909 (MDS Systems) and EP3093, SP186, Bu32, SP199, 1F8 and LT21 (Abcam).

### CEACAM5

Carcinoembryonic antigen-related cell adhesion molecule 5 (CEACAM5) is a member of the carcinoembryonic antigen (CEA) gene family; a set of highly related glycoproteins involved in cell adhesion. CEACAM5 is produced in gastrointestinal tissue during fetal development but is also expressed by some cancers including lung, pancreatic, cervical and gastrointestinal cancers.

CEACAM5 is composed of 642 amino acids, hasa molecular mass of approximately 70 kDa and has 28 potential N-linked glycosylation sites. The protein comprises an Ig variable region (IgV)-like domain, termed N, followed by six Ig constant region (IgC)-type 2-like domains, termed A1, B1, A2, B2, A3, and B3.

Commercially available monoclonal antibodies against CEACAM5 are known, such as EPR20721 (Abcam).

### MUC1

Mucin 1 or MUC1 is a glycoprotein with extensive O-linked glycosylation of its extracellular domain. Mucins line the apical surface of epithelial cells in the lungs, stomach, intestines, eyes and several other organs. They protect the body from infection by pathogen binding to oligosaccharides in the extracellular domain, preventing the pathogen from reaching the cell surface. Overexpression of MUC1 is often associated with colon, breast, ovarian, lung and pancreatic cancers.

MUC1 has a core protein mass of 120-225 kDa which increases to 250-500 kDa with glycosylation. It extends 200-500 nm beyond the surface of the cell. The extracellular domain includes a 20 amino acid variable number tandem repeat (VNTR) domain, with the number of repeats varying from 20 to 120 in different individuals. The most frequent alleles contains 41 and 85 repeats. These repeats are rich in serine, threonine and proline residues which permits heavy o-glycosylation.

Commercially available monoclonal antibodies against MUC1 are known, such as EPR1203, EP1024Y, HMFG1, NCRC48, SM3, MH1 and 115D8 (Abcam).

### FCRL5

Fc receptor-like protein 5 (FCRL5) is a member of the immunoglobulin receptor superfamily and the Fc-receptor like family FCRL5 is a single-pass type I membrane protein and contains 8 immunoglobulin-like C2-type domains. The mature protein is 106 kDa.

FCRL5 has a cytoplasmic tail with two inhibitory ITIM phosphorylation signaling motifs. It inhibits B cell antigen receptor signaling by recruiting SHP1 upon B cell antigen receptor co-stimulation, resulting in diminished calcium influx and protein tyrosine phosphorylation. Co-stimulation of FCRL5 and the B cell antigen receptor promotes proliferation and differentiation of naive B cells. FCRL5 is expressed on both mature B cells and plasma cells, and is induced by EBV proteins. It is overexpressed on malignant B cells of hairy cell leukemia, chronic lymphocytic leukemia, mantle cell lymphoma, and multiple myeloma patients.

Commercially available monoclonal antibodies against FCRL5 are known, such as CD307e (ThermoFisher) and REA391 (Miltenyi Biotec).

The present inventors have also generated four new anti-FCRL5 antibodies, the VH, VL and CDR sequences of which are summarised in Table 2. The CDR sequences are in bold an underlined.

TABLE 2

| Antibody | VH | VL |
|---|---|---|
| 1F6 | QVQLKESGPGLVQPSQTLSLTC | DIQMTQSPSVLSASVGDRVTLS |
| | TVSGFSLTSYTVSWVRQPPGKG | CKASQNINKNLDWYQQKLGEA |
| | LEWIAAISSGGSTYYNSALKSRL | PKWYFTNNLQTGIPSRFSGSG |
| | SISRDTSKSQVFLKMNSLQTEDT | SGTDYTLTISSLQPEDVATYYCY |
| | AMYFCARYTTDSGFDYWGQGV | QYNSGWTFGGGTKLELK |
| | MVTVSS (SEQ ID No. 107) | (SEQ ID No. 108) |
| 2H9 | EVQLVESGGDLVQPGRSLKLSC | DIQMTQSPASLSASLGETVTIEC |
| | ASSGFTFSDYNMAWVRQAPKK | RASEDIYNGLTWYQQKPGKSP |
| | GLEWVATISYDGTNTYYRDSVK | QLLISNANCLHTGVPSRFSGSG |
| | GRFTISRDNAKSTLYLQMDSLR | SGTQYSLKINSLQSEDVASYFC |
| | SEDTATYYCARQDSSYVYLSWF | QQYYNYPWTFGGGTKLDLK |
| | AYWGQGTLVTVSS (SEQ ID No. | (SEQ ID No. 110) |
| | 109) | |
| 7F10 | QVQLKESGPGLVQPSQTLSLTC | DIQMTQSPPILSASVGDRVTLSC |
| | TVSGFSLTSYTVSWVRQPPGR | KASQNINKNLDWYQQKHGEAP |
| | GLEWIAAISSGGNTYYNSGLKS | KLLIYYTHNLQTGIPSRFSGSGS |
| | RLSISRDTSKSQVFLKMNSLQTE | GTDYTLTISSLQPEDVATYYCYQ |
| | DTAMYFCARYAQIRGKDYWGQ | YYSGWTFGGGTKLQLK |
| | GVMVTVSS (SEQ ID No. 111) | (SEQ ID No. 112) |

### OR GATES

The CAR of the present invention may be used in a combination with one or more other activatory or inhibitory chimeric antigen receptors. For example, they may be used in combination with one or more other CARs in a “logic-gate”, a CAR combination which, when expressed by a cell, such as a T cell, are capable of detecting a particular pattern of expression of at least two target antigens. If the at least two target antigens are arbitrarily denoted as antigen A and antigen B, the three possible options are as follows:
“OR GATE”—T cell triggers when either antigen A or antigen B is present on the target cell
“AND GATE”—T cell triggers only when both antigens A and B are present on the target cell
“AND NOT GATE”—T cell triggers if antigen A is present alone on the target cell, but not if both antigens A and B are present on the target cell

Engineered T cells expressing these CAR combinations can be tailored to be exquisitely specific for cancer cells, based on their particular expression (or lack of expression) of two or more markers.

Such “Logic Gates” are described, for example, in WO2015/075469, WO2015/075470 and WO2015/075470.

An “OR Gate” comprises two or more activatory CARs each directed to a distinct target antigen expressed by a target cell. The advantage of an OR gate is that the effective targetable antigen is increased on the target cell, as it is effectively antigen A+antigen B. This is especially important for antigens expressed at variable or low density on the target cell, as the level of a single antigen may be below the threshold needed for effective targeting by a CAR-T cell. Also, it avoids the phenomenon of antigen escape. For example, some lymphomas and leukemias become CD19 negative after CD19 targeting: using an OR gate which targets CD19 in combination with another antigen provides a “back-up” antigen, should this occur.

The FabCAR of the present invention may be used in an OR gate in combination with a second CAR against a second target antigen expressed by the target cell.

For an anti-CD22 FabCAR, the OR gate may comprise a CAR against a second antigen expressed in B cells, such as CD19, CD20 or CD79.

The second CAR may have any suitable antigen binding domain, for example a binding domain based on an scFv, a domain antibody (dAb) or a Fab.

The second CAR may comprise a spacer to spatially separate the antigen binding domain from the transmembrane domain and provide a degree of flexibility. A variety of sequences are commonly used as spacers for CAR, for example, an IgG1 Fc region, an IgG1 hinge (as described above) or a human CD8 stalk. The spacer may comprise a coiled-coil domain, for example as described in WO2016/151315.

The second CAR comprises an activating endodomain. It may, for example comprise the endodomain from CD3ζ. It may comprise one or more co-stimulatory domains as described above. For example, it may comprise the endodomains from CD28, OX-40 or 4-1BB.

The FabCAR of the present invention may be used in a triple OR gate, which comprises a second CAR against a second antigen and a third CAR against a third antigen expressed by the target cell.

For an anti-CD22 FabCAR, a triple OR gate may comprise CARs against second and third antigens expressed in B cells, such as CD19, CD20 or CD79.

In particular, the present invention provides a triple OR gate which comprises:
(i) an anti-CD22 FabCAR;
(ii) an anti-CD79 dAb CAR; and
(iii) an anti-CD19 scFv CAR (see FIG. 7b).

### Dual Fab CARs

The OR gate of the present invention may comprise two (or more) Fab CARs.

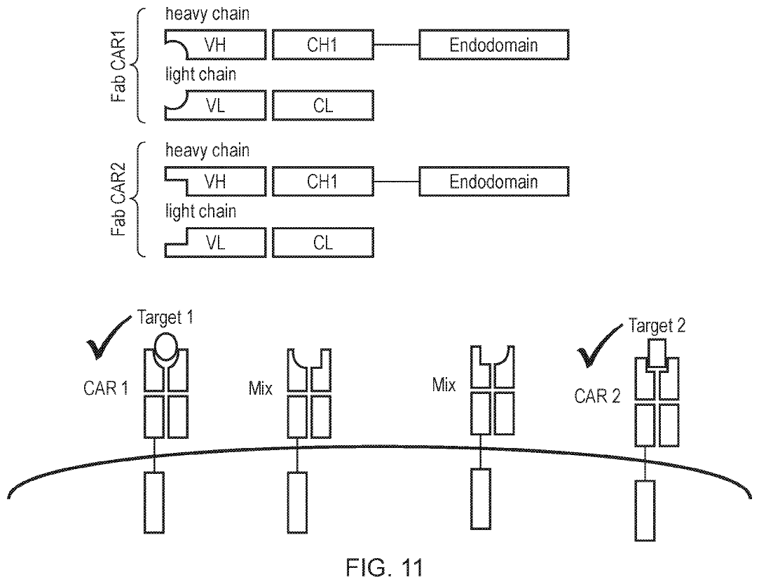

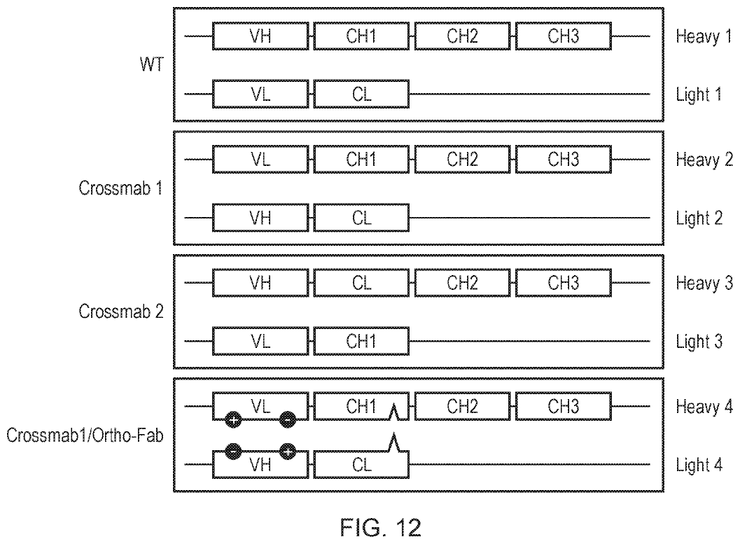

A problem associated with the expression of two Fab CARs is cross pairing or mis-pairing events, creating non-functional CARs (FIG. 11). In order to avoid this “Crossmab” and/or “Ortho-Fab” formats may be used, as illustrated schematically in FIG. 12.

“Crossmab” involves switching the CL and CH1 domains between chains so that a variable light chain (VL) is connected to a heavy chain constant domain (CH) in one molecule; and that a variable heavy chain (VH) is connected to a light chain constant domain (CL) in the other molecule (FIG. 12, Crossmab 1 and 2).

A nucleic acid construct encoding a FabCAR in a crossmab format may have the structure:

VH-CL-spacer-TM-endo-coexpr-VL-CH or

VL-CH-spacer-TM-endo-coexpr-VH-CL
in which:
VH is a nucleic acid sequence encoding a heavy chain variable region;
CH is a nucleic acid sequence encoding a heavy chain constant region spacer is a nucleic acid encoding a spacer;
TM is a a nucleic acid sequence encoding a transmembrane domain;
endo is a nucleic acid sequence encoding an endodomain;
coexpr is a nucleic acid sequence enabling co-expression of the first and second polypeptides;
VL is a nucleic acid sequence encoding a light chain variable region; and
CL is a nucleic acid sequence encoding a light chain constant region.

“Ortho-Fab” involves introducing mutations to avoid alternative combinations. For example, amino acids with bulky side chains may be engineered into one chain (e.g. CL) to create a protrusion and the correctly pairing domain (e.g. CH) may be engineered to accommodate the protrusion. Alternatively, or in addition electrically charged side chains may be engineered into one chain (e.g. VH engineered to have positively charged amino acid) and the correctly pairing domain (e.g. VL) be engineered to have a negatively charges amino acid.

A Dual FabCAR of the invention may comprise a CD19 Fab CAR having the CDRs shown as SEQ ID No. 69-74 in a wild-type Fab CAR format; and a CD22 Fab CAR having the CDRs shown as SEQ ID No. 93-98 in an orthoFab or crossmab1 Fab CAR format.

### CD79 Binders

The term “CD79” or “Cluster of differentiation 79” refers to the protein at the surface of B cells that encompasses two transmembrane proteins, CD79a and CD79b, which form a disulfide-linked heterodimer and are members of the immunoglobulin (Ig) gene superfamily. The transmembrane CD79a and CD79b proteins couple at the extracellular end with any one of the five different types of transmembrane Ig molecules (IgM, IgD, IgG, IgE, or IgA), which are disulfide-linked proteins composed of two Ig heavy chains and two Ig light chains. This combination of CD79 and immunoglobulin on the B-cell surface forms the B-cell signalling receptor (BCR). The intracytoplasmic domains of CD79a and CD79b contain immunoreceptor tyrosine-based activation motifs (ITAMs) that transmit activation signals to the B-cell upon antigen-induced BCR aggregation.

CD79 expression is restricted to Pre-B cells and mature B cells (excluding plasma cells). CD79 is also expressed on a majority of B-cell-derived malignancies. This narrow expression pattern makes it a promising target for cancer-targeted therapies with minimal targeting to normal tissue.

The term “CD79a” or “CD79A” refers to the B-cell antigen receptor complex-associated protein alpha chain also known as Ig-alpha, MB-1 membrane glycoprotein, membrane-bound immunoglobulin-associated protein, and surface IgM-associated protein. The human isoforms of CD79a are depicted under Accession Nos. P11912.1 (Isoform 1 or long) and P11912.2 (Isoform 2 or short) in the Uniprot database on 20 Apr. 2018.

The term “CD79b” or “CD79B” refers to the B-cell antigen receptor complex-associated protein beta chain also known as Ig-beta, B-cell-specific glycoprotein B29, and immunoglobulin-associated B29 protein. The human isoforms of CD79b are depicted under Accession Nos. P40259-1 (Isoform long), P40259-2 (Isoform short) and P40259-3 (Isoform 3) in the Uniprot database on 20 Apr. 2018.

Activated B lymphocytes have increased amounts of the short or truncated CD79 isoforms. In a particular embodiment, the invention relates to a CAR which specifically binds CD79a. In a preferred embodiment, the CAR binds the unspliced portion or CD79a ectodomain, i.e. residues 33 to 143 of CD79a isoform 1, shown below as SEQ ID No. 67 (Uniprot Accession No. P11912.1). In another particular embodiment, the invention relates to a CAR which specifically binds CD79b. In another preferred embodiment, the CAR binds the unspliced portion or CD79b ectodomain, i.e. residues 29 to 159 of CD79b isoform long, shown below as SEQ ID No. 68 (Uniprot Accession No. P40259-1).

CD79a isoform 1
SEQ ID No. 67
MPGGPGVLQALPATIFLLFLLSAVYLGPGCQALWMHKVPASLMVSLG
EDAHFQCPHNSSNNANVTWWRVLHGNYTWPPEFLGPGEDPNGTLIIQ
NVNKSHGGIYVCRVQEGNESYQQSCGTYLRVRQPPPRPFLDMGEGTK
NRIITAEGIILLFCAVVPGTLLLFRKRWQNEKLGLDAGDEYEDENLY
EGLNLDDCSMYEDISRGLQGTYQDVGSLNIGDVQLEKP
CD79b isoform 2
SEQ ID No. 68
MARLALSPVPSHWMVALLLLLSAEPVPAARSEDRYRNPKGSACSRIW
QSPRFIARKRGFTVKMHCYMNSASGNVSWLWKQEMDENPQQLKLEKG
RMEESQNESLATLTIQGIRFEDNGIYFCQQKCNNTSEVYQGCGTELR
VMGFSTLAQLKQRNTLKDGIIMIQTLLIILFIIVPIFLLLDKDDSKA
GMEEDHTYEGLDIDQTATYEDIVTLRTGEVKWSVGEHPGQE

The invention provides OR gates comprising a chimeric antigen receptor (CAR) which binds CD79.

The CAR may specifically bind CD79A. For example, it may bind the unspliced portion of CD79A ectodomain (residues 33 to 143 of SEQ ID NO: 67).

The CAR may specifically bind CD79B. For example it may bind the unspliced portion of CD79B ectodomain (residues 29 to 159 of SEQ ID NO: 68).

Numerous anti-CD79 antibodies are known in the art, for example JCB117, SN8, CB3.1 and 2F2 (Polatuzumab).

The CD79-binding domain may comprise
a) a heavy chain variable region (VH) having complementarity determining regions (CDRs) with the following sequences:

CDR1
(SEQ ID No. 5)
SDYAWN;
CDR2
(SEQ ID No. 6)
NIWYSGSTTYNPSLKS
CDR3
(SEQ ID No. 7)
MDF;

and
b) a light chain variable region (VL) having CDRs with the following sequences:

CDR1
(SEQ ID No. 8)
RASESVDSYGKTFMHWH;
CDR2
(SEQ ID No. 9)
RVSNLES
CDR3
(SEQ ID No. 10)
QQSNEDPFT.

The anti-CD79 CAR may comprise the following VH sequence:

VH sequence from murine monoclonal antibody
SEQ ID No. 11
EVQLQESGPGLVKPSQSLSLTCTVTGYSITSDYAWNWIRQFPGNKLEW
MGNIWYSGSTTYNPSLKSRISITRDTSKNQFFLQLNSVTSEDTATYYC
SRMDFWGQGTTLTVSS

The anti-CD79 CAR may comprise the following VL sequence:

VL sequence from murine monoclonal antibody
SEQ ID No 12
DIVLTQSPPSLAVSLGQRATISCRASESVDSYGKTFMHWHQQKPGQPP
KLLIYRVSNLESGIPARFSGSGSRTDFTLTINPVEADDVATYYCQQSN
EDPFTFGGGTKLEIKR

The anti-CD79 CAR may comprise the following scFv sequence:

(mouse anti-cynomolgus (Macaca fascicularis)
CD79b 10D10 scFv)
SEQ ID NO: 13
DIVLTQSPPSLAVSLGQRATISCRASESVDSYGKTFMHWHQQKPGQPP
KLLIYRVSNLESGIPARFSGSGSRTDFTLTINPVEADDVATYYCQQSN
EDPFTFGGGTKLEIKRSGGGGSGGGGSGGGGSGGGGSEVQLQESGPGL
VKPSQSLSLTCTVTGYSITSDYAWNWIRQFPGNKLEWMGNIWYSGSTT
YNPSLKSRISITRDTSKNQFFLQLNSVTSEDTATYYCSRMDFWGQGTT
LTVSS

Alternatively, the anti-CD79 CAR may comprise an antigen-binding domain which comprises a) a heavy chain variable region (VH) having complementarity determining regions (CDRs) with the following sequences:

CDR1
(SEQ ID No. 14)
SYWIE;
CDR2
(SEQ ID No. 15)
EILPGGGDTNYNEIFKG
CDR3
(SEQ ID No. 16)
RVPVYFDY;

and
b) a light chain variable region (VL) having CDRs with the following sequences:

CDR1
(SEQ ID No. 17)
KASQSVDYDGDSFLN;
CDR2
(SEQ ID No. 18)
AASNLES
CDR3
(SEQ ID No. 19)
QQSNEDPLT.

The anti-CD79 CAR may comprise the following VH sequence:

VH sequence from murine monoclonal antibody
SEQ ID No. 20
EVQLVESGGGLVQPGGSLRLSCAASGYTFSSYWIEWVRQAPGKGLEWI
GEILPGGGDTNYNEIFKGRATFSADTSKNTAYLQMNSLRAEDTAVYYC
TRRVPVYFDYWGQGTLVTVSS

The anti-CD79 CAR may comprise the following VL sequence:

VL sequence from murine monoclonal antibody
SEQ ID No 21
DIQLTQSPSSLSASVGDRVTITCKASQSVDYDGDSFLNWYQQKPGKAP
KLLIYAASNLESGVPSRFSGSGSGTDFTLTISSLQPEDFATYYCQQSN
EDPLTFGQGTKVEIKR

The anti-CD79 CAR may comprise the following scFv sequence:

(humanised anti-CD79b-v17 scFv)
SEQ ID NO: 22
METDTLLLWVLLLWVPGSTGDIQLTQSPSSLSASVGDRVTITCKASQS
VDYDGDSFLNWYQQKPGKAPKLLIYAASNLESGVPSRFSGSGSGTDFT
LTISSLQPEDFATYYCQQSNEDPLTFGQGTKVEIKRSGGGGSGGGGSG
GGGSGGGGSEVQLVESGGGLVQPGGSLRLSCAASGYTFSSYWIEWVRQ
APGKGLEWIGEILPGGGDTNYNEIFKGRATFSADTSKNTAYLQMNSLR
AEDTAVYYCTRRVPVYFDYWGQGTLVTVSS

Alternatively, the anti-CD79 CAR may comprise an antigen-binding domain which comprises a) a heavy chain variable region (VH) having complementarity determining regions (CDRs) with the following sequences:

CDR1
(SEQ ID No. 14)
SYWIE;
CDR2
(SEQ ID No. 15)
EILPGGGDTNYNEIFKG
CDR3
(SEQ ID No. 23)
RVPIRLDY;

and
b) a light chain variable region (VL) having CDRs with the following sequences:

CDR1
(SEQ ID No. 17)
KASQSVDYDGDSFLN;
CDR2
(SEQ ID No. 18)
AASNLES
CDR3
(SEQ ID No. 19)
QQSNEDPLT.

The anti-CD79 CAR may comprise the following VH sequence:

SEQ ID No. 24 - VH sequence from murine monoclonal
antibody
EVQLVESGGGLVQPGGSLRLSCAASGYTFSSYWIEWVRQAPGKGLEWIGE
ILPGGGDTNYNEIFKGRATFSADTSKNTAYLQMNSLRAEDTAVYYCTRRV
PIRLDYWGQGTLVTVSS

The anti-CD79 CAR may comprise the following VL sequence:

VL sequence from murine monoclonal antibody
SEQ ID No. 21
DIQLTQSPSSLSASVGDRVTITCKASQSVDYDGDSFLNVVYQQKPGKAPK
LLIYAASNLESGVPSRFSGSGSGTDFTLTISSLQPEDFATYYCQQSNEDP
LTFGQGTKVEIKR

The anti-CD79 CAR may comprise the following scFv sequence:

(humanised anti-CD79b v18 scFv)
SEQ ID NO: 25
DIQLTQSPSSLSASVGDRVTITCKASQSVDYDGDSFLNVVYQQKPGKAPK
LLIYAASNLESGVPSRFSGSGSGTDFTLTISSLQPEDFATYYCQQSNEDP
LTFGQGTKVEIKRSGGGGSGGGGSGGGGSGGGGSEVQLVESGGGLVQPGG
SLRLSCAASGYTFSSYWIEVVVRQAPGKGLEWIGEILPGGGDTNYNEIFK
GRATFSADTSKNTAYLQMNSLRAEDTAVYYCTRRVPIRLDYWGQGTLVTV
SS

Alternatively, the anti-CD79 CAR may comprise an antigen-binding domain which comprises a) a heavy chain variable region (VH) having complementarity determining regions (CDRs) with the following sequences:

CDR1 -
(SEQ ID No. 14)
SYWIE;
CDR2 -
(SEQ ID No. 15)
EILPGGGDTNYNEIFKG
CDR3 -
(SEQ ID No. 23)
RVPIRLDY;

and
b) a light chain variable region (VL) having CDRs with the following sequences:

CDR1 -
(SEQ ID No. 26)
KASQSVDYEGDSFLN;
CDR2 -
(SEQ ID No. 18)
AASNLES
CDR3 -
(SEQ ID No. 19)
QQSNEDPLT.

The anti-CD79 CAR may comprise the following VH sequence:

SEQ ID No. 24 - VH sequence from murine monoclonal
antibody
EVQLVESGGGLVQPGGSLRLSCAASGYTFSSYWIEWVRQAPGKGLEWIGE
ILPGGGDTNYNEIFKGRATFSADTSKNTAYLQMNSLRAEDTAVYYCTRRV
PIRLDYWGQGTLVTVSS

The anti-CD79 CAR may comprise the following VL sequence:

VL sequence from murine monoclonal antibody
SEQ ID No. 27
DIQLTQSPSSLSASVGDRVTITCKASQSVDYEGDSFLNVVYQQKPGKAPK
LLIYAASNLESGVPSRFSGSGSGTDFTLTISSLQPEDFATYYCQQSNEDP
LTFGQGTKVEIKR

The anti-CD79 CAR may comprise the following scFv sequence:

(humanised anti-CD79b v28 scFv)
SEQ ID NO: 28
METDTLLLWVLLLVVVPGSTGDIQLTQSPSSLSASVGDRVTITCKASQS
VDYEGDSFLNWYQQKPGKAPKLLIYAASNLESGVPSRFSGSGSGTDFTL
TISSLQPEDFATYYCQQSNEDPLTFGQGTKVEIKRSGGGGSGGGGSGGGG
SGGGGSEVQLVESGGGLVQPGGSLRLSCAASGYTFSSYWIEVVVRQAPGK
GLEWIGEILPGGGDTNYNEIFKGRATFSADTSKNTAYLQMNSLRAEDTAV
YYCTRRVPIRLDYWGQGTLVTVSS

Alternatively, the anti-CD79 CAR may comprise an antigen-binding domain which comprises a) a heavy chain variable region (VH) having complementarity determining regions (CDRs) with the following sequences:

CDR1 -
(SEQ ID No. 14)
SYWIE;
CDR2 -
(SEQ ID No. 15)
EILPGGGDTNYNEIFKG
CDR3 -
(SEQ ID NO: 23)
RVPIRLDY;

and
b) a light chain variable region (VL) having CDRs with the following sequences:

CDR1 -
(SEQ ID No. 29)
KASQSVDYSGDSFLN;
CDR2 -
(SEQ ID No. 18)
AASNLES
CDR3 -
(SEQ ID No. 19)
QQSNEDPLT

The anti-CD79 CAR may comprise the following VH sequence:

SEQ ID No. 24 - VH sequence from murine monoclonal
antibody
EVQLVESGGGLVQPGGSLRLSCAASGYTFSSYWIEWVRQAPGKGLEWIGE
ILPGGGDTNYNEIFKGRATFSADTSKNTAYLQMNSLRAEDTAVYYCTRRV
PIRLDYWGQGTLVTVSS

The anti-CD79 CAR may comprise the following VL sequence:

VL sequence from murine monoclonal antibody
SEQ ID No. 30
DIQLTQSPSSLSASVGDRVTITCKASQSVDYSGDSFLNWYQQKPGKAPK
LFIYAASNLESGVPSRFSGSGSGTDFTLTISSLQPEDFATYYCQQSNEDP
LTFGQGTKVEIKR

The anti-CD79 CAR may comprise the following scFv sequence:

(humanised anti-CD79b v32 scFv)
SEQ ID NO: 31
METDTLLLWVLLLVVVPGSTGDIQLTQSPSSLSASVGDRVTITCKASQS
VDYSGDSFLNWYQQKPGKAPKLFIYAASNLESGVPSRFSGSGSGTDFTL
TISSLQPEDFATYYCQQSNEDPLTFGQGTKVEIKRSGGGGSGGGGSGGGG
SGGGGSEVQLVESGGGLVQPGGSLRLSCAASGYTFSSYWIEWVRQAPGK
GLEWIGEILPGGGDTNYNEIFKGRATFSADTSKNTAYLQMNSLRAEDTAV
YYCTRRVPIRLDYWGQGTLVTVSS

Alternatively, the anti-CD79 CAR may comprise an antigen-binding domain which comprises a) a heavy chain variable region (VH) having complementarity determining regions (CDRs) with the following sequences:

CDR1 -
(SEQ ID No. 14)
SYWIE;
CDR2 -
(SEQ ID No. 15)
EILPGGGDTNYNEIFKG
CDR3 -
(SEQ ID NO: 16)
RVPVYFDY;

and
b) a light chain variable region (VL) having CDRs with the following sequences:

CDR1 -
(SEQ ID No. 17)
KASQSVDYDGDSFLN;
CDR2 -
(SEQ ID No. 18)
AASNLES
CDR3 -
(SEQ ID No. 19)
QQSNEDPLT.

The anti-CD79 CAR may comprise the following VH sequence:

SEQ ID No. 32 - VH sequence from murine monoclonal
antibody
EVQLQQSGAELMKPGASVKISCKATGYTFSSYWIEWVKQRPGHGLEWIGE
ILPGGGDTNYNEIFKGKATFTADTSSNTAYMQLSSLTSEDSAVYYCTRRV
PVYFDWGQGTSVTVSS

The anti-CD79 CAR may comprise the following VL sequence:

VL sequence from murine monoclonal antibody
SEQ ID No. 33
DIVLTQSPASLAVSLGQRATISCKASQSVDYDGDSF
LNVVYQQKPGQPPKLFIYAASNLESGIPARFSGSGS
GTDFTLNIHPVEEEDAATYYCQQSNEDPLTFGAGTE
LELKR

The anti-CD79 CAR may comprise the following scFv sequence:

(murine anti-CD79b SN8 scFv)
SEQ ID NO: 34
METDTLLLWVLLLWVPGSTGDIVLTQSPASLAVSL
GQRATISCKASQSVDYDGDSFLNWYQQKPGQPPKL
FIYAASNLESGIPARFSGSGSGTDFTLNIHPVEEE
DAATYYCQQSNEDPLTFGAGTELELKRSGGGGSGG
GGSGGGGSGGGGSEVQLQQSGAELMKPGASVKISC
KATGYTFSSYWIEWVKQRPGHGLEWIGEILPGGGD
TNYNEIFKGKATFTADTSSNTAYMQLSSLTSEDSA
VYYCTRRVPVYFDYWGQGTSVTVSS

Alternatively, the anti-CD79 CAR may comprise an antigen-binding domain which comprises a) a heavy chain variable region (VH) having complementarity determining regions (CDRs) with the following sequences:

CDR1
(SEQ ID No. 14)
SYWIE;
CDR2
(SEQ ID No. 15)
EILPGGGDTNYNEIFKG
CDR3
(SEQ ID NO: 23)
RVPIRLDY;

and
b) a light chain variable region (VL) having CDRs with the following sequences:

CDR1
(SEQ ID No. 26)
KASQSVDYEGDSFLN;
CDR2
(SEQ ID No. 18)
AASNLES
CDR3
(SEQ ID No. 19)
QQSNEDPLT.

The anti-CD79 CAR may comprise the following VH sequence:

SEQ ID No. 24 - VH sequence from murine monoclonal
antibody
EVQLVESGGGLVQPGGSLRLSCAASGYTFSSYWIEWVRQAPGKGLEWIGE
ILPGGGDTNYNEIFKGRATFSADTSKNTAYLQMNSLRAEDTAVYYCTRRV
PIRLDYWGQGTLVTVSS

The anti-CD79 CAR may comprise the following VL sequence:

VL sequence from murine monoclonal antibody
SEQ ID No. 27
DIQLTQSPSSLSASVGDRVTITCKASQSVDYEGDSFLNWY
QQKPGKAPKLLIYAASNLESGVPSRFSGSGSGTDFTLTISS
LQPEDFATYYCQQSNEDPLTFGQGTKVEIKR

The anti-CD79 CAR may comprise the following scFv sequence:

(humanised anti-CD79b 2F2 scFv)
SEQ ID NO: 28
METDTLLLWVLLLWVPGSTGDIQLTQSPSSLSASVGDRVTITC
KASQSVDYEGDSFLNWYQQKPGKAPKLLIYAASNLESGVPSRF
SGSGSGTDFTLTISSLQPEDFATYYCQQSNEDPLTFGQGTKVE
IKRSGGGGSGGGGSGGGGSGGGGSEVQLVESGGGLVQPGGSLR
LSCAASGYTFSSYWIEWVRQAPGKGLEWIGEILPGGGDTNYNE
IFKGRATFSADTSKNTAYLQMNSLRAEDTAVYYCTRRVPIRLD
YWGQGTLVTVSS

Alternatively, the anti-CD79 CAR may comprise an antigen-binding domain which comprises a) a heavy chain variable region (VH) having complementarity determining regions (CDRs) with the following sequences:

CDR1
(SEQ ID No. 15)
NYGMN;
CDR2
(SEQ ID No. 16)
RIYPGSGSTNYQKFKG
CDR3
(SEQ ID NO: 35)
YAMDY;

and
b) a light chain variable region (VL) having CDRs with the following sequences:

CDR1
(SEQ ID No. 18)
RSSQSIVHSNGNTYLE;
CDR2
(SEQ ID No. 19)
KVSNRPS
CDR3
(SEQ ID No. 20)
FQGSHVPWT.

The anti-CD79 CAR may comprise the following VH sequence:

SEQ ID No. 36 - VH sequence from murine monoclonal
antibody
QVQLQQSGPELVKPGASVKISCKASGYTFTNYGMNWVKQRPGQGLQWIGR
IYPGSGSTNYQKFKGKATLTVDKSSSTAYMELRSLTSENSAVYYCARYAM
DYTGQGTSVTVSS

The anti-CD79 CAR may comprise the following VL sequence:

VL sequence from murine monoclonal antibody
SEQ ID No. 37
DVLMTQTPLSLPVSLGDQASISCRSSQSIVHSNGNTYLEWYLQKP
GQSPKLLIYKVSNRPSGVPNRFSGSGSGTDFTLKISRVQAQNLGV
YYCFQGSHVPWTFGGGTKLEIKR

The anti-CD79 CAR may comprise the following scFv sequence:

(murine anti-CD79a scFv)
SEQ ID NO: 38
METDTLLLWVLLLWVPGSTGDVLMTQTPLSLPVSLGDQASISCR
SSQSIVHSNGNTYLEWYLQKPGQSPKLLIYKVSNRPSGVPNRFS
GSGSGTDFTLKISRVQAQNLGVYYCFQGSHVPWTFGGGTKLEIK
RSGGGGSGGGGSGGGGSGGGGSQVQLQQSGPELVKPGASVKISC
KASGYTFTNYGMNWVKQRPGQGLQWIGRIYPGSGSTNYQKFKGK
ATLTVDKSSSTAYMELRSLTSENSAVYYCARYAMDYTGQGTSVT
VSS

It may be possible to introduce one or more mutations (substitutions, additions or deletions) into the or each CDR without negatively affecting CD79-binding activity. Each CDR may, for example, have one, two or three amino acid mutations.

### CD19 Binders

Several anti-CD19 antibodies have been previously described in a CAR format, such as fmc63, 4G7, SJ25C1, CAT19 (as described in WO2016/139487) and CD19ALAb (as described in WO2016/102965)

An anti-CD19 CAR for use in a double or triple OR gate of the present invention may comprise an antigen-binding domain, such as an scFv-type antigen binding domain, derived from one of these anti-CD19 antibodies.

The CD19-binding domain may comprises
a) a heavy chain variable region (VH) having complementarity determining regions (CDRs) with the following sequences:

(SEQ ID No. 69)
CDR1 - GYAFSSS;
(SEQ ID No. 70)
CDR2 - YPGDED
(SEQ ID No. 71)
CDR3 - SLLYGDYLDY;

and
b) a light chain variable region (VL) having CDRs with the following sequences:

(SEQ ID No. 72)
CDR1 - SASSSVSYMH;
(SEQ ID No. 73)
CDR2 - DTSKLAS
(SEQ ID No. 74)
CDR3 - QQWNINPLT.

It may be possible to introduce one or more mutations (substitutions, additions or deletions) into each CDR without negatively affecting CD19-binding activity. Each CDR may, for example, have one, two or three amino acid mutations.

The CDRs may be in the format of a single-chain variable fragment (scFv), which is a fusion protein of the heavy variable region (VH) and light chain variable region (VL) of an antibody, connected with a short linker peptide of ten to about 25 amino acids. The scFv may be in the orientation VH-VL, i.e. the VH is at the amino-terminus of the CAR molecule and the VL domain is linked to the spacer and, in turn the transmembrane domain and endodomain.

The CDRs may be grafted on to the framework of a human antibody or scFv. For example, the CAR of the present invention may comprise a CD19-binding domain consisting or comprising one of the following sequences

The anti-CD19 CAR may comprise the following VH sequence:

SEQ ID No. 75 - VH sequence from murine monoclonal
antibody
QVQLQQSGPELVKPGASVKISCKASGYAFSSSWMNWVKQRPGKGLEWIG
RIYPGDEDTNYSGKFKDKATLTADKSSTTAYMQLSSLTSEDSAVYFCAR
SLLYGDYLDYWGQGTTLTVSS

The anti-CD19 CAR may comprise the following VL sequence:

VL sequence from murine monoclonal antibody
SEQ ID No 76
QIVLTQSPAIMSASPGEKVTMTCSASSSVSYMHWYQQKSGTSPKRWIYD
TSKLASGVPDRFSGSGSGTSYFLTINNMEAEDAATYYCQQWNINPLTFG
AGTKLELKR

The anti-CD19 CAR may comprise the following scFv sequence:

VH-VL scFv sequence from murine monoclonal
antibody
SEQ ID No 77
QVQLQQSGPELVKPGASVKISCKASGYAFSSSWMNWVKQRPGKGLEWIG
RIYPGDEDTNYSGKFKDKATLTADKSSTTAYMQLSSLTSEDSAVYFCAR
SLLYGDYLDYWGQGTTLTVSSGGGGSGGGGSGGGGSQIVLTQSPAIMSA
SPGEKVTMTCSASSSVSYMHWYQQKSGTSPKRWIYDTSKLASGVPDRFS
GSGSGTSYFLTINNMEAEDAATYYCQQWNINPLTFGAGTKLELKR

Alternatively, the anti-CD19 CAR may comprise an antigen-binding domain which comprises a) a heavy chain variable region (VH) having complementarity determining regions (CDRs) with the following sequences:

(SEQ ID No. 78)
CDR1 - SYWMN;
(SEQ ID No. 79)
CDR2 - QIWPGDGDTNYNGKFK
(SEQ ID No. 80)
CDR3 - RETTTVGRYYYAMDY;

and
b) a light chain variable region (VL) having CDRs with the following sequences:

(SEQ ID No. 81)
CDR1 - KASQSVDYDGDSYLN;
(SEQ ID No. 82)
CDR2 - DASNLVS
(SEQ ID No. 83)
CDR3 - QQSTEDPWT.

It may be possible to introduce one or more mutations (substitutions, additions or deletions) into the or each CDR without negatively affecting CD19-binding activity. Each CDR may, for example, have one, two or three amino acid mutations.

The CAR of the present invention may comprise one of the following amino acid sequences:

(Murine CD19ALAb scFv sequence)
SEQ ID No. 84
QVQLQQSGAELVRPGSSVKISCKASGYAFSSYWMNWVKQRPGQGLEWI
GQIWPGDGDTNYNGKFKGKATLTADESSSTAYMQLSSLASEDSAVYFC
ARRETTTVGRYYYAMDYWGQGTTVTVSSDIQLTQSPASLAVSLGQRAT
ISCKASQSVDYDGDSYLNWYQQIPGQPPKLLIYDASNLVSGIPPRFSG
SGSGTDFTLNIHPVEKVDAATYHCQQSTEDPWTFGGGTKLEIK
(Humanised CD19ALAb scFv sequence - Heavy 19,
Kappa 16)
SEQ ID No. 85
QVQLVQSGAEVKKPGASVKLSCKASGYAFSSYWMNWVRQAPGQSLEWI
GQIWPGDGDTNYNGKFKGRATLTADESARTAYMELSSLRSGDTAVYFC
ARRETTTVGRYYYAMDYWGKGTLVTVSSDIQLTQSPDSLAVSLGERAT
INCKASQSVDYDGDSYLNWYQQKPGQPPKLLIYDASNLVSGVPDRFSG
SGSGTDFTLTISSLQAADVAVYHCQQSTEDPWTFGQGTKVEIKR
(Humanised CD19ALAb scFv sequence - Heavy 19,
Kappa 7)
SEQ ID No. 86
QVQLVQSGAEVKKPGASVKLSCKASGYAFSSYWMNWVRQAPGQSLEWI
GQIWPGDGDTNYNGKFKGRATLTADESARTAYMELSSLRSGDTAVYFC
ARRETTTVGRYYYAMDYWGKGTLVTVSSDIQLTQSPDSLAVSLGERAT
INCKASQSVDYDGDSYLNWYQQKPGQPPKVLIYDASNLVSGVPDRFSG
SGSGTDFTLTISSLQAADVAVYYCQQSTEDPWTFGQGTKVEIKR

The scFv may be in a VH-VL orientation (as shown in SEQ ID No.s 84, 85 and 86) or a VL-VH orientation.

The CAR of the present invention may comprise one of the following VH sequences:

(Murine CD19ALAb VH sequence)
SEQ ID No. 87
QVQLQQSGAELVRPGSSVKISCKASGYAFSSYWMNWVKQRPGQGLEWI
GQIWPGDGDTNYNGKFKGKATLTADESSSTAYMQLSSLASEDSAVYFC
ARRETTTVGRYYYAMDYWGQGTTVTVSS
(Humanised CD19ALAb VH sequence)
SEQ ID No. 88
QVQLVQSGAEVKKPGASVKLSCKASGYAFSSYWMNWVRQAPGQSLEWI
GQIWPGDGDTNYNGKFKGRATLTADESARTAYMELSSLRSGDTAVYFC
ARRETTTVGRYYYAMDYWGKGTLVTVSS

An anti-CD19 CAR may comprise one of the following VL sequences:

(Murine CD19ALAb VL sequence)
SEQ ID No. 89
DIQLTQSPASLAVSLGQRATISCKASQSVDYDGDSYLNWYQQIPGQPP
KLLIYDASNLVSGIPPRFSGSGSGTDFTLNIHPVEKVDAATYHCQQST
EDPWTFGGGTKLEIK
(Humanised CD19ALAb VL sequence, Kappa 16)
SEQ ID No. 90
DIQLTQSPDSLAVSLGERATINCKASQSVDYDGDSYLNWYQQKPGQPP
KWYDASNLVSGVPDRFSGSGSGTDFTLTISSLQAADVAVYHCQQSTED
PWTFGQGTKVEIKR
(Humanised CD19ALAb VL sequence, Kappa 7)
SEQ ID No. 91
DIQLTQSPDSLAVSLGERATINCKASQSVDYDGDSYLNWYQQKPGQPPK
VLIYDASNLVSGVPDRFSGSGSGTDFTLTISSLQAADVAVYYCQQSTED
PWTFGQGTKVEIKR

The CAR may comprise a variant of the sequence shown as SEQ ID No. 84 to 91having at least 80, 85, 90, 95, 98 or 99% sequence identity, provided that the variant sequence retain the capacity to bind CD19 (when in conjunction with a complementary VL or VH domain, if appropriate).

The percentage identity between two polypeptide sequences may be readily determined by programs Is BLAST which is freely available at http://blast.ncbi.nlm.nih.gov.

### Nucleic Acid Construct

The present invention also provides a nucleic acid construct encoding a chimeric receptor of the invention.

A nucleic acid construct encoding a FabCAR (FIG. 6A) may have the structure:

VH-CH-spacer-TM-endo-coexpr-VL-CL or

VL-CL-spacer-TM-endo-coexpr-VH-CH
in which:
VH is a nucleic acid sequence encoding a heavy chain variable region;
CH is a nucleic acid sequence encoding a heavy chain constant region spacer is a nucleic acid encoding a spacer;
TM is a a nucleic acid sequence encoding a transmembrane domain;
endo is a nucleic acid sequence encoding an endodomain;
coexpr is a nucleic acid sequence enabling co-expression of the first and second polypeptides;
VL is a nucleic acid sequence encoding a light chain variable region; and
CL is a nucleic acid sequence encoding a light chain constant region.

For both structures mentioned above, nucleic acid sequences encoding the two polypeptides may be in either order in the construct.

There is also provided a nucleic acid construct encoding an OR gate, which comprises two of more CARs, at least one of which is a FabCAR according to the present invention.

A nucleic acid construct encoding a double OR gate may have the structure:

VH-CH-spacer1-TM1-endo1-coexpr1-VL-CL-coexpr2-AgBD-spacer2-TM2-endo2; or

VL-CL-spacer-TM1-endo1-coexpr1-VH-CH-coexpr2-AgBD-spacer2-TM2-endo2
in which:
VH is a nucleic acid sequence encoding a heavy chain variable region of the first CAR;
CH is a nucleic acid sequence encoding a heavy chain constant region of the first CAR;
Spacer 1 is a nucleic acid sequence encoding a spacer of the first CAR;
TM1 is a a nucleic acid sequence encoding a transmembrane domain of the first CAR;
Endo1 is a nucleic acid sequence encoding an endodomain of the first CAR;
Coexpr1 and coexpr2, which my be the same or different, are nucleic acid sequences enabling co-expression of the first and second polypeptides of the first CAR; and the first and second CARs;
VL is a nucleic acid sequence encoding a light chain variable region of the first CAR;
CL is a nucleic acid sequence encoding a light chain constant region of the first CAR;
AgBD is a nucleic acid sequence encoding an antigen binding domain of the second CAR;
Spacer2 is a nucleic acid sequence encoding a spacer of the second CAR;
TM2 is a a nucleic acid sequence encoding a transmembrane domain of the second CAR; and
Endo2 is a nucleic acid sequence encoding an endodomain of the second CAR.

The antigen-binding domain of the second CAR may, for example, be an scFv or a dAb.

For both structures mentioned above, nucleic acid sequences encoding the two polypeptides of the first CAR; and the nucleic acid sequences encoding the first and second CARs may be in any order in the construct.

There is also provided a nucleic acid construct encoding a triple OR gate, which comprises three CARs, one of which is a FabCAR according to the present invention.

A nucleic acid construct encoding a triple OR gate may have the structure:

VH-CH-spacer1-TM1-endo1-coexpr1-VL-CL-coexpr2-AgBD2-spacer2-TM2-endo2-coexpr3-AgBD3-spacer3-TM3-endo3; or

VL-CL-spacer1-TM1-endo1-coexpr1-VH-CH-coexpr2-AgBD2-spacer2-TM2-endo2-coexpr3-AgBD3-spacer3-TM3
in which:
VH is a nucleic acid sequence encoding a heavy chain variable region of the first CAR;
CH is a nucleic acid sequence encoding a heavy chain constant region of the first CAR;
Spacer 1 is a nucleic acid sequence encoding a spacer of the first CAR;
TM1 is a a nucleic acid sequence encoding a transmembrane domain of the first CAR;
Endo1 is a nucleic acid sequence encoding an endodomain of the first CAR;
Coexpr1, coexpr2 and coexpr3, which my be the same or different, are nucleic acid sequences enabling co-expression of the first and second polypeptides of the first CAR; and the first, second and third CARs;
VL is a nucleic acid sequence encoding a light chain variable region of the first CAR;
CL is a nucleic acid sequence encoding a light chain constant region of the first CAR;
AgBD2 is a nucleic acid sequence encoding an antigen binding domain of the second CAR;
Spacer2 is a nucleic acid sequence encoding a spacer of the second CAR;
TM2 is a a nucleic acid sequence encoding a transmembrane domain of the second CAR;
Endo2 is a nucleic acid sequence encoding an endodomain of the second CAR;
AgBD3 is a nucleic acid sequence encoding an antigen binding domain of the third CAR;
Spacer3 is a nucleic acid sequence encoding a spacer of the third CAR;
TM3 is a a nucleic acid sequence encoding a transmembrane domain of the third CAR; and
Endo3 is a nucleic acid sequence encoding an endodomain of the third CAR;

The antigen-binding domain of the second and third CARs may, for example, be an scFv or a dAb. In particular, one CAR may have a dAb antigen-binding domain and the other may have an scFv antigen binding domain.

In particular, the construct may be as illustrated in FIG. 7a. The construct may encode three CARs as illustrated in FIG. 7b, namely a FabCAR against CD22; a dAb CAR against CD79 and an scFV CAR against CD19.

As used herein, the terms “polynucleotide”, “nucleotide”, and “nucleic acid” are intended to be synonymous with each other.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed.

Nucleic acids according to the invention may comprise DNA or RNA. They may be single-stranded or double-stranded. They may also be polynucleotides which include within them synthetic or modified nucleotides. A number of different types of modification to oligonucleotides known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

In the structure above, “coexpr” is a nucleic acid sequence enabling co-expression of two polypeptides as separate entities. It may be a sequence encoding a cleavage site, such that the nucleic acid construct produces both polypeptides, joined by a cleavage site(s). The cleavage site may be self-cleaving, such that when the polypeptide is produced, it is immediately cleaved into individual peptides without the need for any external cleavage activity.

The cleavage site may be any sequence which enables the two polypeptides to become separated.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may, for example be a furin cleavage site, a Tobacco Etch Virus (TEV) cleavage site or encode a self-cleaving peptide.

A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho-or a cardiovirus. The primary 2A/2B cleavage of the aptho-and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho-or cardioviruses, ‘picornavirus-like’ insect viruses, type C rotaviruses and repeated sequences within Trypanosoma spp and a bacterial sequence (Donnelly et al (2001) as above).

The cleavage site may comprise the 2A-like sequence shown as SEQ ID No.92 (RAEGRGSLLTCGDVEENPGP).

### Vector

The present invention also provides a vector, or kit of vectors, which comprises one or more nucleic acid sequence(s) encoding a chimeric receptor according to the invention. Such a vector may be used to introduce the nucleic acid sequence(s) into a host cell so that it expresses a chimeric polypeptide according to the first aspect of the invention.

The vector may, for example, be a plasmid or a viral vector, such as a retroviral vector or a lentiviral vector, or a transposon based vector or synthetic mRNA.

The vector may be capable of transfecting or transducing a T cell or a NK cell.

### Cell

The present invention provides a cell which comprises a chimeric antigen receptor of the invention. The cell may comprise two of more CARs, for example it may comprise a double or triple or gate as described above.

The cell may comprise a nucleic acid or a vector of the present invention.

The cell may be a cytolytic immune cell such as a T cell or an NK cell.

T cells or T lymphocytes are a type of lymphocyte that play a central role in cell-mediated immunity. They can be distinguished from other lymphocytes, such as B cells and natural killer cells (NK cells), by the presence of a T-cell receptor (TCR) on the cell surface. There are various types of T cell, as summarised below.

Helper T helper cells (TH cells) assist other white blood cells in immunologic processes, including maturation of B cells into plasma cells and memory B cells, and activation of cytotoxic T cells and macrophages. TH cells express CD4 on their surface. TH cells become activated when they are presented with peptide antigens by MHC class II molecules on the surface of antigen presenting cells (APCs). These cells can differentiate into one of several subtypes, including TH1, TH2, TH3, TH17, Th9, or TFH, which secrete different cytokines to facilitate different types of immune responses.

Cytolytic T cells (TC cells, or CTLs) destroy virally infected cells and tumor cells, and are also implicated in transplant rejection. CTLs express the CD8 at their surface. These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO.

Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described—naturally occurring Treg cells and adaptive Treg cells.

Naturally occurring Treg cells (also known as CD4+CD25+FoxP3+ Treg cells) arise in the thymus and have been linked to interactions between developing T cells with both myeloid (CD11c+) and plasmacytoid (CD123+) dendritic cells that have been activated with TSLP. Naturally occurring Treg cells can be distinguished from other T cells by the presence of an intracellular molecule called FoxP3. Mutations of the FOXP3 gene can prevent regulatory T cell development, causing the fatal autoimmune disease IPEX.

Adaptive Treg cells (also known as Tr1 cells or Th3 cells) may originate during a normal immune response.

The cell may be a Natural Killer cell (or NK cell). NK cells form part of the innate immune system. NK cells provide rapid responses to innate signals from virally infected cells in an MHC independent manner

NK cells (belonging to the group of innate lymphoid cells) are defined as large granular lymphocytes (LGL) and constitute the third kind of cells differentiated from the common lymphoid progenitor generating B and T lymphocytes. NK cells are known to differentiate and mature in the bone marrow, lymph node, spleen, tonsils and thymus where they then enter into the circulation.

The cells of the invention may be any of the cell types mentioned above.

T or NK cells according to the first aspect of the invention may either be created ex vivo either from a patient's own peripheral blood (1st party), or in the setting of a haematopoietic stem cell transplant from donor peripheral blood (2nd party), or peripheral blood from an unconnected donor (3rd party).

Alternatively, T or NK cells according to the first aspect of the invention may be derived from ex vivo differentiation of inducible progenitor cells or embryonic progenitor cells to T or NK cells. Alternatively, an immortalized T-cell line which retains its lytic function and could act as a therapeutic may be used.

In all these embodiments, chimeric polypeptide-expressing cells are generated by introducing DNA or RNA coding for the chimeric polypeptide by one of many means including transduction with a viral vector, transfection with DNA or RNA.

The cell of the invention may be an ex vivo T or NK cell from a subject. The T or NK cell may be from a peripheral blood mononuclear cell (PBMC) sample. T or NK cells may be activated and/or expanded prior to being transduced with nucleic acid encoding the molecules providing the chimeric polypeptide according to the first aspect of the invention, for example by treatment with an anti-CD3 monoclonal antibody.

The T or NK cell of the invention may be made by:
(i) isolation of a T or NK cell-containing sample from a subject or other sources listed above; and
(ii) transduction or transfection of the T or NK cells with one or more a nucleic acid sequence(s) encoding a chimeric polypeptide.

The T or NK cells may then by purified, for example, selected on the basis of expression of the antigen-binding domain of the antigen-binding polypeptide.

### Pharmaceutical Composition

The present invention also relates to a pharmaceutical composition containing a plurality of cells according to the invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Method of Treatment

The present invention provides a method for treating and/or preventing a disease which comprises the step of administering the cells of the present invention (for example in a pharmaceutical composition as described above) to a subject.

A method for treating a disease relates to the therapeutic use of the cells of the present invention. Herein the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. Herein such cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a T or NK cell-containing sample;
(ii) transducing or transfecting such cells with a nucleic acid sequence or vector provided by the present invention;
(iii) administering the cells from (ii) to a subject.

The T or NK cell-containing sample may be isolated from a subject or from other sources, for example as described above. The T or NK cells may be isolated from a subject's own peripheral blood (1st party), or in the setting of a haematopoietic stem cell transplant from donor peripheral blood (2nd party), or peripheral blood from an unconnected donor (3rd party).

The present invention provides a chimeric polypeptide-expressing cell of the present invention for use in treating and/or preventing a disease.

The invention also relates to the use of a chimeric polypeptide-expressing cell of the present invention in the manufacture of a medicament for the treatment and/or prevention of a disease.

The disease to be treated and/or prevented by the methods of the present invention may be a cancerous disease, such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The disease may be Multiple Myeloma (MM), B-cell Acute Lymphoblastic Leukaemia (B-ALL), Chronic Lymphocytic Leukaemia (CLL), Neuroblastoma, T-cell acute Lymphoblastic Leukaema (T-ALL) or diffuse large B-cell lymphoma (DLBCL).

The cells of the present invention may be capable of killing target cells, such as cancer cells. The target cell may be characterised by the presence of a tumour secreted ligand or chemokine ligand in the vicinity of the target cell. The target cell may be characterised by the presence of a soluble ligand together with the expression of a tumour-associated antigen (TAA) at the target cell surface.

The cells and pharmaceutical compositions of present invention may be for use in the treatment and/or prevention of the diseases described above.

### Further Aspects

The present invention also provides a new CD22-binding antibody, termed 9A8-1. The VH, VL and CDR sequences of 9A8 are shown in Table 1 above.

This antibody shows particularly good efficacy in a CAR. For example, as shown in Example 3 below, 9A8-1 in a FabCAR format showed improved target cell killing and cytokine release that an equivalent CAR with an alternative CD22 binder, 3B4.

The present invention also provides the aspects summarised in the following numbered paragraphs.

1. An antigen-binding domain which comprises:
a) a heavy chain variable region (VH) having complementarity determining regions (CDRs) with the following sequences:

(SEQ ID No. 93)
CDR1 - NFAMA
(SEQ ID No. 94)
CDR2 - SISTGGGNTYYRDSVKG
(SEQ ID No. 95)
CDR3 - QRNYYDGSYDYEGYTMDA;

and
b) a light chain variable region (VL) having complementarity determining regions (CDRs) with the following sequences:

(SEQ ID No. 96)
CDR1 - RSSQDIGNYLT
(SEQ ID No. 97)
CDR2 - GAIKLED
(SEQ ID No. 98)
CDR3 - LQSIQYP

2. An antigen-binding domain according to paragraph 1, which comprises a VH domain having the sequence shown as SEQ ID No. 65; and a VL domain having the sequence shown as SEQ ID No. 66.

3. An antibody which comprises an antigen-binding domain according to paragraph 1 or 2.

4. An antibody-drug conjugate (ADC) or bispecific T-cell engager (BiTE) which comprises an antibody according to paragraph 3.

5. A chimeric antigen receptor (CAR) which comprises an antigen-binding domain according to paragraph 1 or 2.

6. A CAR according to paragraph 5, which is a FabCAR.

7. A CAR according to paragraph 5, which is an scFv CAR.

8. A nucleic acid sequence which encodes an antigen-binding domain according to paragraph 1 or 2, and antibody according to paragraph 3, an ADC or BiTE according to paragraph 4, or a CAR according to any of paragraphs 5 to 7.

9. A nucleic acid sequence according to claim 8 which encodes a CAR according to any of paragraphs 5 to 7 and has a GC content of at least 60%.

10. A nucleic acid sequence according to claim 8 which encodes a CAR according to any of paragraphs 5 to 7 and has a GC content of about 64%.

11. A nucleic acid sequence according to any of claims 8 to 10 which comprises an elongation factor-1 alpha (EF1α) promoter.

12. A nucleic acid construct which comprises a first nucleic acid sequence according to any of claims 8 to 11 encoding a CAR according to any of paragraphs 5 to 7 and a second nucleic acid sequence encoding an anti-CD19 CAR.

13. A nucleic acid construct according to claim 12, wherein the antigen binding domain of the anti-CD19 CAR comprises
a) a heavy chain variable region (VH) having complementarity determining regions (CDRs) with the following sequences:

(SEQ ID No. 69)
CDR1 - GYAFSSS;
(SEQ ID No. 70)
CDR2 - YPGDED
(SEQ ID No. 71)
CDR3 - SLLYGDYLDY;

and
b) a light chain variable region (VL) having CDRs with the following sequences:

(SEQ ID No. 72)
CDR1 - SASSSVSYMH;
(SEQ ID No. 73)
CDR2 - DTSKLAS
(SEQ ID No. 74)
CDR3 - QQWNINPLT.

14. A nucleic acid construct according to claim 13, wherein the antigen-binding domain of the anti-CD19 CAR comprises a VH domain as shown in SEQ ID No. 75 and a VL domain as shown as SEQ ID No. 76.

15. A nucleic acid construct according to any of claims 12 to 14, wherein the anti-CD22 CAR is in a Fab format, the nucleic acid construct having the general structure:

VH-CH-spacer1-TM1-endo1-coexpr1-VL-CL-coexpr2-AgBD-spacer2-TM2-endo2;

VL-CL-spacer-TM1-endo1-coexpr1-VH-CH-coexpr2-AgBD-spacer2-TM2-endo2;

AgBD-spacer2-TM2-endo2-VH-CH-spacer1-TM1-endo1-coexpr2-VL-CL;

AgBD-spacer2-TM2-endo2-coexpr1-VL-CL-spacer-TM1-endo1-coexpr2-VH-CH VL-CL-coexpr1-VH-CH-spacer1-TM1-endo1--coexpr2-AgBD-spacer2-TM2-endo2;

VH-CH-coexpr1-VL-CL-spacer-TM1-endo1-coexpr2-AgBD-spacer2-TM2-endo2;

AgBD-spacer2-TM2-endo2-VL-CL-coexpr2-VH-CH-spacer1-TM1-endo1; or

AgBD-spacer2-TM2-endo2-coexpr1-VH-CH-coexpr2-VL-CL-spacer-TM1-endo1
in which:
VH is a nucleic acid sequence encoding a heavy chain variable region of the first CAR;
CH is a nucleic acid sequence encoding a heavy chain constant region of the first CAR;
Spacer 1 is a nucleic acid sequence encoding a spacer of the first CAR;
TM1 is a a nucleic acid sequence encoding a transmembrane domain of the first CAR;
Endo1 is a nucleic acid sequence encoding an endodomain of the first CAR;
Coexpr1 and coexpr2, which my be the same or different, are nucleic acid sequences enabling co-expression of the first and second polypeptides of the first CAR; and the second CAR;
VL is a nucleic acid sequence encoding a light chain variable region of the first CAR;
CL is a nucleic acid sequence encoding a light chain constant region of the first CAR;
AgBD is a nucleic acid sequence encoding an antigen binding domain of the second CAR;
Spacer2 is a nucleic acid sequence encoding a spacer of the second CAR;
TM2 is a a nucleic acid sequence encoding a transmembrane domain of the second CAR; and
Endo2 is a nucleic acid sequence encoding an endodomain of the second CAR.

16. A nucleic acid construct according to any of claims 12 to 14, wherein the anti-CD22 CAR is in an ScFv format, the nucleic acid construct having the general structure:

AgBD1-spacer1-TM1-endo1-coexpr-AgBD2-spacer2-TM2-endo2; or

AgBD2-spacer2-TM2-endo2-coexpr-AgBD1-spacer1-TM1-endo1
In which:
AgBD1 is a nucleic acid sequence encoding an antigen binding domain of the first CAR;
Spacer1 is a nucleic acid sequence encoding a spacer of the first CAR;
TM1 is a a nucleic acid sequence encoding a transmembrane domain of the first CAR; and
Endo1 is a nucleic acid sequence encoding an endodomain of the first CAR.
Coexpr is a nucleic acid sequence enabling co-expression of the first and second CARs;
AgBD2 is a nucleic acid sequence encoding an antigen binding domain of the second CAR;
Spacer2 is a nucleic acid sequence encoding a spacer of the second CAR;
TM2 is a a nucleic acid sequence encoding a transmembrane domain of the second CAR; and
Endo2 is a nucleic acid sequence encoding an endodomain of the second CAR.

17. A vector which comprises a nucleic acid sequence according to any of paragraphs 8 to 11 or a nucleic acid construct according to any of paragraphs 12 to 16.

18. A kit of vectors which comprises a first vector comprising a first nucleic acid sequence as defined in any of paragraphs 12 to 16; and a second vector comprising a second nucleic acid sequence as defined in any of paragraphs 12 to 16.

19. A vector or kit of vectors according to paragraph 17 or 18 which is/are retroviral vector(s).

20. A vector or kit of vectors according to paragraph 17 or 18 which is/are lentiviral vector(s).

21. A cell which expresses a CAR according to any of paragraphs 5 to 7.

22. A cell which co-expresses a first CAR according to any of paragraphs 5 to 7 and a second CAR which is an anti-CD19 CAR.

23. A cell according to claim 22, wherein the antigen binding domain of the anti-CD19 CAR comprises
a) a heavy chain variable region (VH) having complementarity determining regions (CDRs) with the following sequences:

(SEQ ID No. 69)
CDR1 - GYAFSSS;
(SEQ ID No. 70)
CDR2 - YPGDED
(SEQ ID No. 71)
CDR3 - SLLYGDYLDY;

and
b) a light chain variable region (VL) having CDRs with the following sequences:

(SEQ ID No. 72)
CDR1 - SASSSVSYMH;
(SEQ ID No. 73)
CDR2 - DTSKLAS
(SEQ ID No. 74)
CDR3 - QQWNINPLT.

24. A cell according to paragraph 23, wherein the antigen-binding domain of the anti-CD19 CAR comprises a VH domain as shown in SEQ ID No. 75 and a VL domain as shown as SEQ ID No. 76.

25. A method for making a cell according to any of paragraphs 21 to 24, which comprises the step of introducing a CAR-encoding nucleic acid sequence according to any of paragraphs 8 to 11; a nucleic-acid construct according to any of paragraphs 12 to 16 or a vector or kit of vectors according to any of paragraphs 17 to 20 into a cell ex vivo.

26. A pharmaceutical composition which comprises a plurality of cells according to any of paragraphs 21 to 24, together with a pharmaceutically acceptable carrier, diluent or excipient.

27. A method for treating cancer which comprises the step of administering a pharmaceutical composition according to paragraph 26 to a subject.

28. A method according to paragraph 27, wherein the cancer is a B-cell leukemia or lymphoma.

29. A cell according to any of paragraphs 21 to 24 for use in treating a cancer.

30. The use of a cell according to any of paragraphs 21 to 24 in the manufacture of a pharmaceutical composition for treating cancer.

General features of, for example, chimeric antigen receptors, nucleic acid sequences and constructs, vectors, cells, pharmaceutical compositions and method of making and using cells described in the preceding sections also apply to the corresponding components described in the paragraphs above.

The present invention also provides a new CD22-binding antibody, termed 1G3-4. The VH, VL and CDR sequences of 1G3-4 are shown in Table 1 above.

This antibody shows particularly good efficacy in a CAR. For example, as shown in Example 3 below, 9A8-1 in a FabCAR format showed improved target cell killing and cytokine release that an equivalent CAR with an alternative CD22 binder, 3B4.

The present invention also provides the aspects summarised in the following numbered paragraphs.

A1. An antigen-binding domain which comprises:
a) a heavy chain variable region (VH) having complementarity determining regions (CDRs) with the following sequences:

(SEQ ID No. 101)
CDR1 - TSGMGVG
(SEQ ID No. 102)
CDR2 - NIWWDDDKNYNPSLKN
(SEQ ID No. 103)
CDR3 - IAHYFDGYYYVMDV;

and
b) a light chain variable region (VL) having complementarity determining regions (CDRs) with the following sequences:

(SEQ ID No. 104)
CDR1 - LASGGISNDLA
(SEQ ID No. 105)
CDR2 - AASRLQD
(SEQ ID No. 106)
CDR3 - QQSYKYPY

A2. An antigen-binding domain according to paragraph A1, which comprises a VH domain having the sequence shown as SEQ ID No. 99; and a VL domain having the sequence shown as SEQ ID No. 100.

A3. An antibody which comprises an antigen-binding domain according to paragraph A1 or A2.

A4. An antibody-drug conjugate (ADC) or bispecific T-cell engager (BiTE) which comprises an antibody according to paragraph A3.

A5. A chimeric antigen receptor (CAR) which comprises an antigen-binding domain according to paragraph A1 or A2.

A6. A CAR according to paragraph A5, which is a FabCAR.

A7. A nucleic acid sequence which encodes an antigen-binding domain according to paragraph A1 or A2, and antibody according to paragraph A3, an ADC or BiTE according to paragraph A4, or a CAR according to paragraph A5 or A6.

A8. A vector which comprises a nucleic acid sequence according to paragraph A7.

A9. A cell which expresses a CAR according to paragraph A5 or A6.

A10. A method for making a cell according to paragraph A9, which comprises the step of introducing a CAR-encoding nucleic acid sequence according to paragraph A7 into a cell.

A11. A pharmaceutical composition which comprises a plurality of cells according to paragraph A9, together with a pharmaceutically acceptable carrier, diluent or excipient.

A12. A method for treating cancer which comprises the step of administering a pharmaceutical composition according to paragraph A11 to a subject.

A13. A method according to paragraph A12, wherein the cancer is a B-cell leukemia or lymphoma.

A14. A cell according to paragraph A9 for use in treating a cancer.

A15. The use of a cell according to paragraph A9 in the manufacture of a pharmaceutical composition for treating cancer.

B1. An antigen-binding domain which comprises:
ai) a heavy chain variable region (VH) having complementarity determining regions (CDRs) with the following sequences:

(SEQ ID No. 113)
CDR1 - SYTVS
(SEQ ID No. 114)
CDR2 - AISSGGSTYYNSALKS
(SEQ ID No. 115)
CDR3 - YTTDSGFDY;

and
bi) a light chain variable region (VL) having complementarity determining regions (CDRs) with the following sequences:

(SEQ ID No. 116)
CDR1 - KASQNINKNLD
(SEQ ID No. 117)
CDR2 - FTNNLQT
(SEQ ID No. 118)
CDR3 - YQYNSGWT;

or
aii) a heavy chain variable region (VH) having complementarity determining regions (CDRs) with the following sequences:

(SEQ ID No. 119)
CDR1 - DYNMA
(SEQ ID No. 120)
CDR2 - TISYDGTNTYYRDSVKG
(SEQ ID No. 121)
CDR3 - QDSSYVYLSWFAY;

and
bii) a light chain variable region (VL) having complementarity determining regions (CDRs) with the following sequences:

(SEQ ID No. 122)
CDR1 - RASEDIYNGLT
(SEQ ID No. 123)
CDR2 - NANCLHT
(SEQ ID No. 124)
CDR3 - QQYYNYPWT;

or
aiii) a heavy chain variable region (VH) having complementarity determining regions (CDRs) with the following sequences:

(SEQ ID No. 125)
CDR1 - SYTVS
(SEQ ID No. 126)
CDR2 - AISSGGNTYYNSGLKS
(SEQ ID No. 127)
CDR3 - YAQIRGKDY;

and
biii) a light chain variable region (VL) having complementarity determining regions (CDRs) with the following sequences:

(SEQ ID No. 128)
CDR1 - KASQNINKNLD
(SEQ ID No. 129)
CDR2 - YTHNLQT
(SEQ ID No. 130)
CDR3 - YQYYSGWT.

B2. An antigen-binding domain according to paragraph 1, which comprises: i) a VH domain having the sequence shown as SEQ ID No. 107; and a VL domain having the sequence shown as SEQ ID No. 108; or ii) a VH domain having the sequence shown as SEQ ID No. 109; and a VL domain having the sequence shown as SEQ ID No. 110; or iii) a VH domain having the sequence shown as SEQ ID No. 111; and a VL domain having the sequence shown as SEQ ID No. 112.

B3. An antibody which comprises an antigen-binding domain according to paragraph B1 or B2.

B4. An antibody-drug conjugate (ADC) or bispecific T-cell engager (BiTE) which comprises an antibody according to paragraph B3.

B5. A chimeric antigen receptor (CAR) which comprises an antigen-binding domain according to paragraph B1 or B2.

B6. A CAR according to paragraph B5, which is a FabCAR.

B7. A nucleic acid sequence which encodes an antigen-binding domain according to paragraph B1 or B2, and antibody according to paragraph B3, an ADC or BiTE according to paragraph B4, or a CAR according to paragraph B5 or B6.

B8. A vector which comprises a nucleic acid sequence according to paragraph B7.

B9. A cell which expresses a CAR according to paragraph B5 or B6.

B10. A method for making a cell according to paragraph B9, which comprises the step of introducing a CAR-encoding nucleic acid sequence according to paragraph B7 into a cell.

B11. A pharmaceutical composition which comprises a plurality of cells according to paragraph B9, together with a pharmaceutically acceptable carrier, diluent or excipient.

B12. A method for treating cancer which comprises the step of administering a pharmaceutical composition according to paragraph B11 to a subject.

B13. A method according to paragraph B12, wherein the cancer is a B-cell leukemia or lymphoma.

B14. A cell according to paragraph B9 for use in treating a cancer.

B15. The use of a cell according to paragraph B9 in the manufacture of a pharmaceutical composition for treating cancer.

General features of, for example, chimeric antigen receptors, nucleic acid sequences and constructs, vectors, cells, pharmaceutical compositions and method of making and using cells described in the preceding sections also apply to the corresponding components described in the paragraphs above.

The invention will now be further described by way of Examples, which are meant to serve to assist one of ordinary skill in the art in carrying out the invention and are not intended in any way to limit the scope of the invention.

### EXAMPLES

A series of experiments were conducted to compare the action of a FabCAR with an scFv CAR based on the same antibody against a target cell expressing an antigen with a bulk extracellular domain (CD22). CAR-T cells expressing the two types of CAR were compared in terms of cytotoxicity and proliferation.

### Example 1—FACs-Based Killing (FBK)

A panel of CARs was created as summarised below and their cytotoxic capability was compared against CD22 expressing target cells.
NT: Non-transduced
10C1-D9 Fab: A FabCAR based on the 10C1 mAb

All CARs had a second generation endodomain comprising CD3ζ and a 4-1BB co-stimulatory domain.

T-cells were co-cultured with the CD22-expressing SupT1 target cells at a ratio of 1:1.The assay was carried out in a 96-well plate in 0.2 ml total volume using 5×10⁴ transduced T-cells per well and an equal number of target cells. The co-cultures are set up after being normalised for the transduction efficiency. The FBK was carried out after 24 h of incubation.

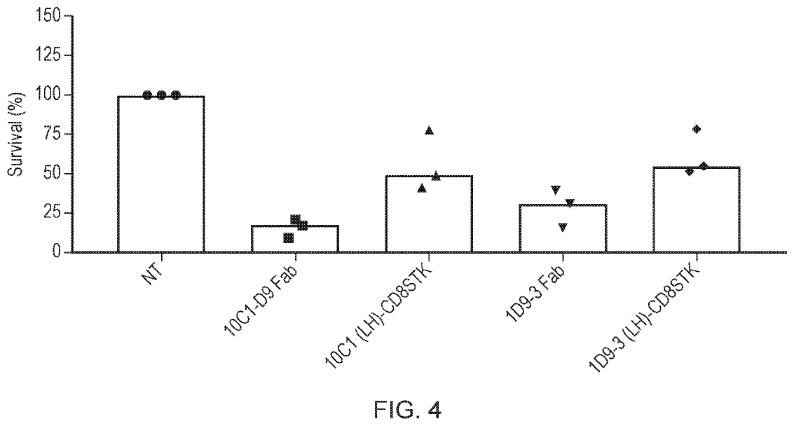

The results of the FBK are shown in FIG. 4. For both CD22 binders tested: 10C1 and 1D9-3, the CAR having a Fab antigen binding domain outperformed the equivalent CAR having an scFv antigen-binding domain in terms of target cell killing.

### Example 2—Proliferation Assay (PA)

In order to measure proliferation the same panel of CAR-expressing T cells described in Example 1 were labelled with the dye Cell Trace Violet (CTV), a fluorescent dye which is hydrolysed and retained within the cell. It is excited by the 405 nm (violet) laser and fluorescence can be detected in the pacific blue channel. The CTV dye was reconstituted to 5 mM in DMSO. The T-cells were resuspended at 2×10⁶ cells per ml in PBS, and 1 ul/ml of CTV was added. The T-cells were incubated the CTV for 20 minutes at 37° C. Subsequently, the cells were quenched by adding 5V of complete media. After a 5 minutes incubation, the T-cells were washed and resuspended in 2 ml of complete media. An additional 10 minute incubation at room temperature allowed the occurrence of acetate hydrolysis and retention of the dye.

Labelled T-cells were co-cultured with Raji target cells for four days. The assay was carried out in a 96-well plate in 0.2 ml total volume using 5×10⁴ transduced T-cells per well and an equal number of target cells (ratio 1:1). At the day four time point, the T-cells were analysed by flow cytometry to measure the dilution of the CTV which occurs as the T-cells divide. The number of T-cells present at the end of the co-culture was calculated, and expressed as a fold of proliferation compared to the input number of T cells.

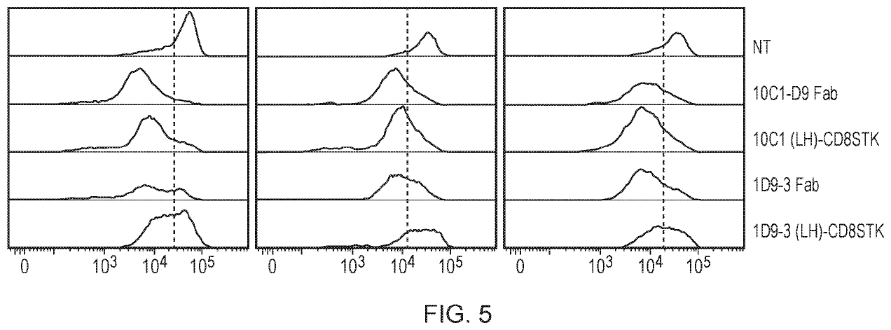

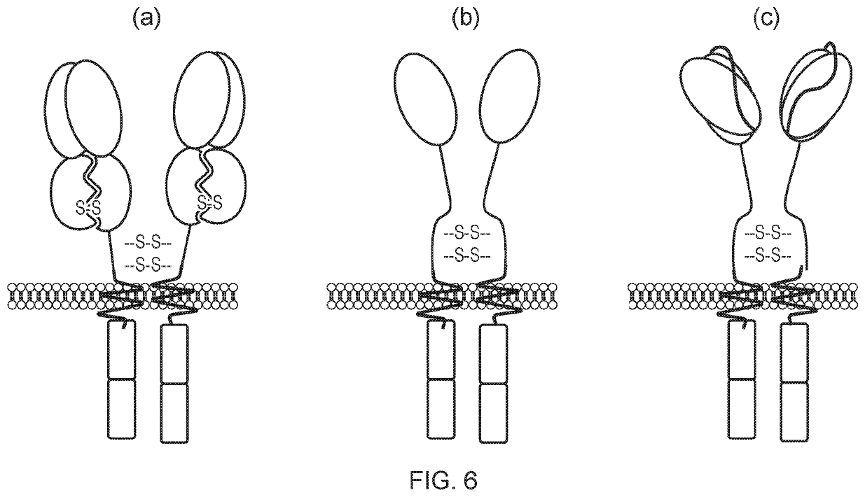

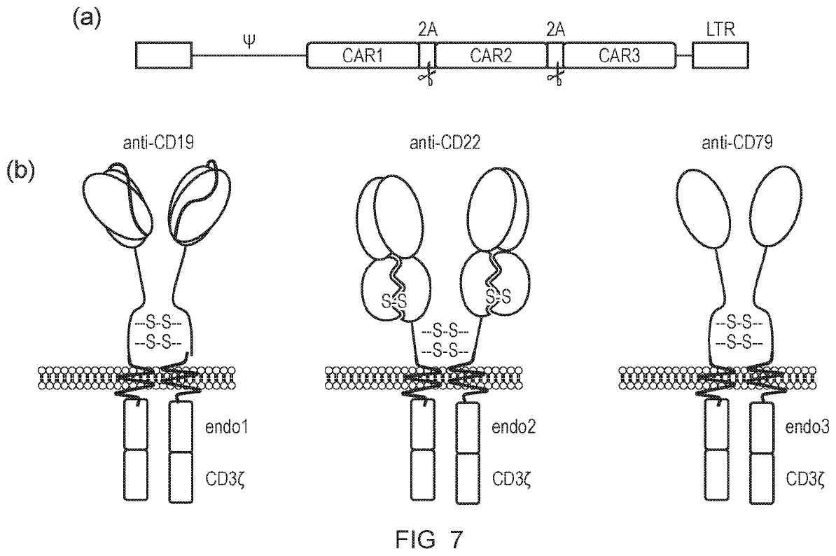

FIG. 5 shows that for both CD22 binders tested: 10C1 and 1D9-3, T cells expressing the CAR having a Fab antigen binding domain proliferated more than the equivalent CAR having an scFv antigen-binding domain. The area under the curve for both both FabCAR constructs has shifted further along the X-axis compared to the equivalent scFvCAR construct.

### Example 3—Investigating the Efficacy of the Anti-CD22 Antibody 9A8-1 in a FabCAR Format

A panel of CARs was created as summarised below and their cytotoxic capability was compared against CD22 expressing SupT1 target cells.
NT: Non-transduced
3B4: A FabCAR based on the 3B4 mAb
9A8: A FabCAR based on the 9A8-1 mAb

All CARs had a second generation endodomain comprising CD3ζ and a 4-1BB co-stimulatory domain.

Firstly, the capacity of T-cells expressing the CARs to kill target cells was investigated. T-cells were co-cultured with the CD22-expressing SupT1 target cells at a ratio of 1:4 E:T. A FACS-based killing assay was carried out after 72 h of incubation as described below.

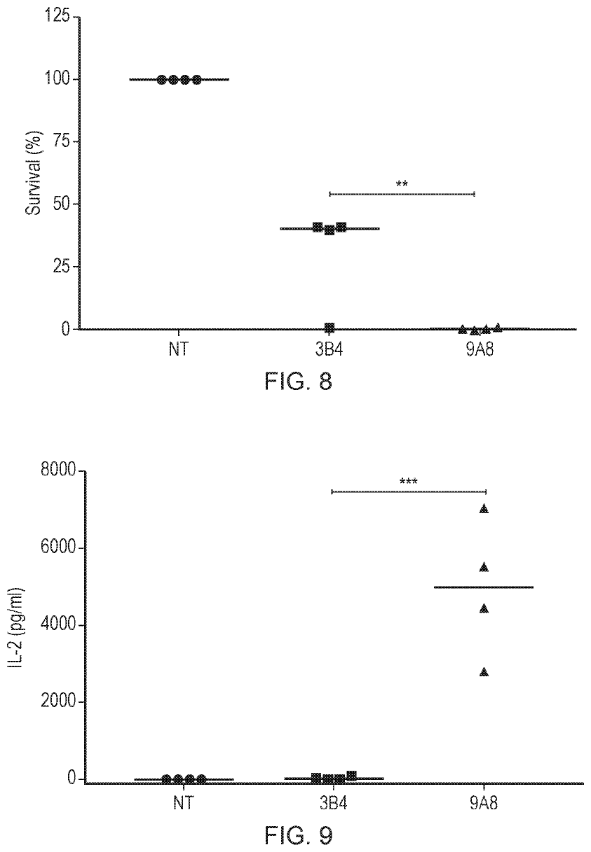

The results of the FBK are shown in FIG. 8. The CAR having a 9A8 antigen-binding domain outperformed the equivalent CAR having a 3B4 antigen-binding domain in terms of target cell killing.

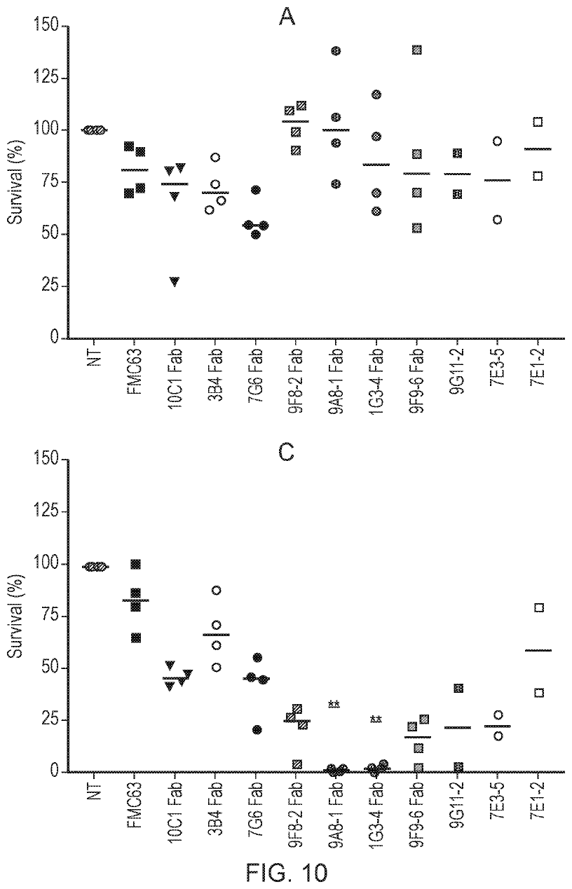

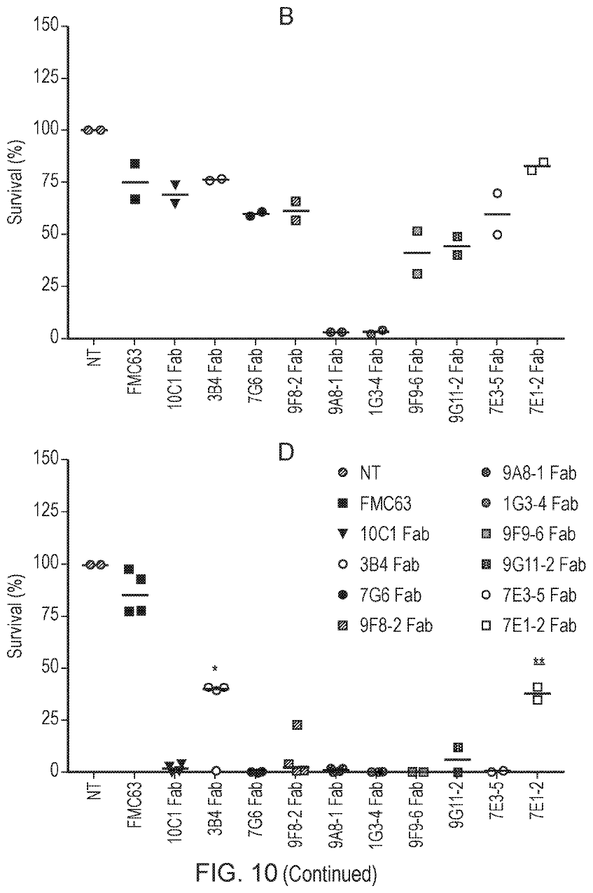

Next, the two CARs were compared in terms of cytokine release. After 72 hr co-culture with CD22-expressing SupT1 target cells, IL-2 expression was investigated by ELISA as described below. The results are shown in FIG. 9. Significantly higher levels of IL-2 release were observed for the CAR having a 9A8 antigen-binding domain than the equivalent CAR having a 3B4 antigen-binding domain.

### Example 4—Investigating the Efficacy of the Anti-CD22 Antibody 1g3-4 in a FabCAR Format

A panel of CARs was created as summarised below:
NT: Non-transduced
Fmc63: An anti-CD19 CAR (negative control)
10C1: An anti-CD22 FabCAR based on the 10C1 mAb
3B4: An anti-CD22 FabCAR based on the 3B4 mAb
7G6: An anti-CD22 FabCAR based on the 7G6 mAb
9F8-2: An anti-CD22 FabCAR based on the 9F8-2 mAb
9A8-1: An anti-CD22 FabCAR based on the 9A8-1 mAb
1G3-4: An anti-CD22 FabCAR based on the 1G3-4 mAb
9F9-6: An anti-CD22 FabCAR based on the 9F9-6 mAb

All CARs had a second generation endodomain comprising CD3ζ and a 4-1BB co-stimulatory domain.

The capacity of T-cells expressing the CARs to kill SupT1 target cells was investigated. SupT1 cells were either left untransduced (FIG. 10, panel A) or transduced to express CD22. Transduced target cells were sorted into three populations: those with a level of CD22 expression which is undetectable by flow cytometry (FIG. 10, panel B); those with a low level of CD22 expression, averaging 255 copies per cell (FIG. 10, panel C); and those with a high level of CD22 expression, averaging 78,916 copies per cell (FIG. 10, panel D).

T-cells were co-cultured with the CD22-expressing SupT1 target cells at a ratio of 1:4 E:T. A FACS-based killing assay was carried out after 72 h of incubation as described below.

The results of the FBK are shown in FIG. 10. All of the anti-CD22 FabCARs showed effective killing of target cells expressing a high level of target antigen (FIG. 10D). However, the Fab CARs having a 9A8 antigen-binding domain or an 1G3-4 antigen binding domain showed killing at of target cells even at an ultra low level of expression of target antigen (panel B).

### Transduction

Retrovirus was generated by transiently transfecting 293T cells using Gene Juice (EMD Millipore) with RDF plasmid (RD114 envelope), gag/pol plasmid and CAR T-cell plasmid and viral supernatant was collected at 48 and 72 hours. T cells were stimulated using 0.5 μg/mL of anti-CD3 and anti-CD28 antibodies in T175 TC-treated flasks and maintained in 100 U/mL IL-2. Non-TC treated six-well plates were coated with Retronectin in accordance to manufacturer's instructions (Takara Bio) and incubated at 4° C. for 24 hours prior to T cell transduction. 3 ml of viral supernatant was plated prior to the addition of 1ml of activated T cells at a concentration of 1×10⁶ cells/ml, 100 U/mL of IL-2 was then added and centrifuged at 1000×g for 40 minutes at room temperature and incubated at 37° C. and 5% CO₂ for 2-3 days.

### NK Cells and NKT Cells Depletion

EasySep™ Human CD56 Positive Selection Kit used to carry out CD56 depletion (STEMCELL 18055).

### Cytotoxicity Assay

To measure cytotoxicity, CAR T-cells were co-cultured with SupT1-NT and SupT1 CD22 at effector: target ratios 4:1 (200,000:50,000 cells) in a TC-treated 96-well plate. Readout was taken at 72 hours by staining with anti-hCD34-APC (FAB7227A), anti-CD2-FITC and anti-CD3-PeCy7 (300419) to differentiate effector T-cells and target cells, 7-AAD cell viability dye (420403) was used to exclude dead cells and phosphate-buffered saline (10010023) to carry out cell washes between incubations. Cytotoxicity readouts were acquired using the MACSQuant® Analyzer 10 flow cytometer (Miltenyi Biotec).

### Cytokine Release

Production of IL-2 by CAR T-cells were measured by collecting supernatant at 72 hr from co-cultures at a 4:1 E: T ratio and freezing at −20° C. prior to analysis by ELISA. Cytokine analysis were performed using the Human IFN-y ELISA MAX™ Deluxe Sets (BioLegend, 430106) and IL-2 ELISA MAX™ Deluxe Sets (BioLegend, 431806) following manufacturers protocol. Varioskan LUX Multimode Microplate Reader (Thermo Fisher) used to measure ELISA signal.

This application claims the benefit of United Kingdom application No. 1807866.7 filed 15 May 2018 and United Kingdom application No. 1809773.3 filed 14 Jun. 2018. Both of the above applications are incorporated herein by reference in their entireties.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1-25. (canceled)

26. A method for killing a CD22-expressing target cell in a subject, the method comprising the step of administering to the subject a cell which expresses a chimeric antigen receptor (CAR) comprising an anti-CD22 antigen-binding domain which comprises:
a) a heavy chain variable region (VH) having complementarity determining regions (CDRs) with the following sequences:
(SEQ ID No. 93)
CDR1 - NFAMA
(SEQ ID No. 94)
CDR2 - SISTGGGNTYYRDSVKG
(SEQ ID No. 95)
CDR3 - QRNYYDGSYDYEGYTMDA;
and
b) a light chain variable region (VL) having complementarity determining regions (CDRs) with the following sequences:
(SEQ ID NO: 96)
CDR1 - RSSQDIGNYLT,
(SEQ ID NO: 97)
CDR2 - GAIKLED,
and
(SEQ ID NO: 98)
CDR3 - LQSIQYP.

27. The method according to claim 26, wherein the antigen-binding domain of the CAR comprises a VH domain having the sequence shown as SEQ ID NO: 65; and a VL domain having the sequence shown as SEQ ID NO: 66.

28. The method according to claim 26, wherein the cell further comprises a second CAR which is an anti-CD19 CAR.

29. The method according to claim 28, wherein the antigen binding domain of the anti-CD19 CAR comprises
a) a heavy chain variable region (VH) having complementarity determining regions (CDRs) with the following sequences:
(SEQ ID NO: 69)
CDR1 - GYAFSSS,
(SEQ ID NO: 70)
CDR2 - YPGDED,
and
(SEQ ID NO: 71)
CDR3 - SLLYGDYLDY,
and
b) a light chain variable region (VL) having CDRs with the following sequences:
(SEQ ID NO: 72)
CDR1 - SASSSVSYMH,
(SEQ ID NO: 73)
CDR2 - DTSKLAS,
and
(SEQ ID NO: 74)
CDR3 - QQWNINPLT.

30. The method according to claim 29, wherein the antigen-binding domain of the anti-CD19 CAR comprises a VH domain as shown in SEQ ID NO: 75 and a VL domain as shown as SEQ ID NO: 76.

31. A method for making a cell, which comprises the step of introducing into a cell a nucleic acid sequence encoding an anti-CD22 chimeric antigen receptor (CAR) which comprises an antigen-binding domain which comprises:
a) a heavy chain variable region (VH) having complementarity determining regions (CDRs) with the following sequences:
(SEQ ID No. 93)
CDR1 - NFAMA
(SEQ ID No. 94)
CDR2 - SISTGGGNTYYRDSVKG
(SEQ ID No. 95)
CDR3 - QRNYYDGSYDYEGYTMDA;
and
b) a light chain variable region (VL) having complementarity determining regions (CDRs) with the following sequences:
(SEQ ID No. 96)
CDR1 - RSSQDIGNYLT
(SEQ ID No. 97)
CDR2 - GAIKLED
(SEQ ID No. 98)
CDR3 - LQSIQYP

32. The method according to claim 31, wherein the antigen-binding domain of the CAR comprises a VH domain having the sequence shown as SEQ ID NO: 65; and a VL domain having the sequence shown as SEQ ID NO: 66.

33. The method according to claim 31, wherein the nucleic acid sequence comprises a first nucleic acid sequence encoding an anti-CD22 CAR and a second nucleic acid sequence encoding an anti-CD19 CAR.

34. The method according to claim 33, wherein the antigen binding domain of the anti-CD19 CAR comprises:
a) a heavy chain variable region (VH) having complementarity determining regions (CDRs) with the following sequences:
(SEQ ID NO: 69)
CDR1 - GYAFSSS,
(SEQ ID NO: 70)
CDR2 - YPGDED,
and
(SEQ ID NO: 71)
CDR3 - SLLYGDYLDY,
and
b) a light chain variable region (VL) having CDRs with the following sequences:
(SEQ ID NO: 72)
CDR1 - SASSSVSYMH,
(SEQ ID NO: 73)
CDR2 - DTSKLAS,
and
(SEQ ID NO: 74)
CDR3 - QQWNINPLT.

35. The method according to claim 34, wherein the antigen-binding domain of the anti-CD19 CAR comprises a VH domain as shown in SEQ ID NO: 75 and a VL domain as shown as SEQ ID NO: 76.

36. The method according to claim 33, wherein the anti-CD22 CAR is in an scFv format, the nucleic acid sequence having the general structure:
AgBD1-spacer1-TM1-endo1-coexpr-AgBD2-spacer2-TM2-endo2 or
AgBD2-spacer2-TM2-endo2-coexpr-AgBD1-spacer1-TM1-endo1,
wherein:
AgBD1 is a nucleic acid sequence encoding an antigen binding domain of the first CAR,
Spacer1 is a nucleic acid sequence encoding a spacer of the first CAR,
TM1 is a nucleic acid sequence encoding a transmembrane domain of the first CAR,
Endo1 is a nucleic acid sequence encoding an endodomain of the first CAR,
Coexpr is a nucleic acid sequence enabling co-expression of the first and second CARS,
AgBD2 is a nucleic acid sequence encoding an antigen binding domain of the second CAR,
Spacer2 is a nucleic acid sequence encoding a spacer of the second CAR,
TM2 is a nucleic acid sequence encoding a transmembrane domain of the second CAR, and
Endo2 is a nucleic acid sequence encoding an endodomain of the second CAR.
